(19) United States
(12) Patent Application Publication
(10) Pub. No.: US 2011/0070269 A1
(43) Pub. Date: Mar. 24, 2011

(54) LIPOPOLYSACCHARIDE ISOLATED FROM PYRULARIA TISSUE AND/OR PYRULARIA-ASSOCIATED BACTERIA AND USES THEREOF

(75) Inventors: STEVEN G. WOOD, OREM, UT (US); KATIE SOUTHWICK, AVENEL, NJ (US); L. DEE NORD, OREM, UT (US)

(73) Assignee: THERAPRO TECHNOLOGIES, INC.

(21) Appl. No.: 12/890,502

(22) Filed: Sep. 24, 2010

Related U.S. Application Data
(60) Provisional application No. 61/245,639, filed on Sep. 24, 2009.

(30) Foreign Application Priority Data
Sep. 24, 2010 (US) PCT/US2010/050133

(51) Int. Cl. A61K 39/00; C07H 15/06; A61K 31/739; A61K 38/19; A61P 35/00

(52) U.S. Cl. 424/277.1; 536/18.2; 514/25; 514/1.1

(57) ABSTRACT

This disclosure provides, in certain embodiments, methods for generating and using extracts of Pyrularia (e.g., Pyrularia pubera) and/or epiphytic/endophytic bacteria associated therewith, either alone or in combination with conventional immunostimulant(s) or anti-inflammatory agents, for modulation of the immune system of a subject. Also provided are compositions that include specific Pyrularia extracts, bacteria isolated from Pyrularia tissue (in particular, Pantoea species), and extracts of from such bacteria. Also provided herein are purified components of Pyrularia extracts and/or extracts from the epiphytic/endophytic bacteria, which extracts exhibit mitogenic or cytotoxic activities. Compositions provided herein can be used for the treatment of neutropenia subsequent to chemotherapy, and for the treatment of immune deficiency (e.g., such as is caused by toxic chemotherapy, disease, or advancing age), as well as in other immune-stimulatory methods. Other embodiments of the invention provide methods of producing extracts of the invention, as well as purified bioactive components thereof, including particularly bacterial LPS.

### CROSS REFERENCE TO RELATED APPLICATIONS

This application claims the benefit of U.S. Provisional Application No. 61/245,639, filed Sep. 24, 2009, and priority to International Application No. PCT/US2010/050133, filed Sep. 24, 2010, both of which are herein incorporated by reference in their entirety.

### FIELD

This disclosure relates to immunologically active components derived from bacteria associated with Pyrularia and methods of use.

### BACKGROUND

Natural protection against host invasion by, for example, bacteria, viruses, or parasites occurs in all higher organisms including insects, plants, and animals. Protection is mediated by biological sensors within the host, which are triggered by binding to specific molecules associated with the invading organism. Recognition then can lead to sequestration of the organisms, or to initiation of processes that lead to the elimination of the invading organism.

For example, in one specific case for human invasion by gram negative bacteria, a portion of the bacterial cell wall known generically as lipopolysaccharide (LPS) can bind to a specific biological sensor known as LPS-binding protein (LBP). The LPS-LBP complex then can be recognized by an immune cell receptor, such as toll-like receptor 4 (TLR-4) on a cell type such as a monocyte or a macrophage, which expresses the receptor. Recognition initiates, via various signal transduction pathways, the synthesis and release of immune mediators (e.g., cytokines and chemokines), which attract, localize, and/or activate specific cell types in a concerted immune response.

In general, LPS has a profound effect upon the mammalian immune system and LPS in high amounts may over-stimulate the host immune system with lethal effect, as in the case of bacteremia leading to septic shock. However, in controlled amounts, LPS is recognized as a potent immune stimulator and has been given systemically without the appearance of clinical symptoms.

### SUMMARY

Disclosed herein is the finding that the Pyrularia plant harbors symbiotic (epiphytic and/or endophytic) bacteria, including Pantoea agglomerans or Pantoea ananatis. Aqueous extracts of plant parts, including root portions and fruit (the fleshy covering of Pyrularia seeds), possess potent immunostimulatory activity, which is dependent on lipopolysaccharide (LPS) of the plant-associated bacteria. Pyrularia plant extracts, and LPS isolated from plant-associated bacteria, possess pronounced immunostimulatory activity resulting in increased numbers of murine or human granulocytes. This activity is reflected in the ability of the extracts to hasten restoration of white blood cell counts and lead to faster bone marrow re-colonization and restoration of blood granulocytes following sub-lethal chemotherapy.

Thus, provided herein are purified immunostimulatory compositions including LPS isolated from Pyrularia fruit extracts, or LPS isolated from a Pantoea species associated with Pyrularia fruit. In some embodiments, the LPS is isolated from Pantoea agglomerans or Pantoea ananatis. Further provided are compositions comprising the Pyrularia fruit extracts, or LPS isolated from a Pantoea species, and a pharmaceutically acceptable carrier.

Also provided herein is a method of stimulating immunity, comprising administering to a subject in need thereof a composition that includes LPS isolated from Pyrularia fruit extracts or a Pantoea species, such as Pantoea agglomerans or Pantoea ananatis. In some embodiments, the method further comprises administering to the subject a therapeutically effective amount of a second immunostimulatory composition, such as a composition comprising a cytokine. In some embodiments, the subject has, or is at risk of developing a tumor. Treatment of the subject with a composition comprising LPS isolated from Pyrularia fruit extracts or a Pantoea species can inhibit tumor development in the subject, such as inhibit metastasis of the tumor.

Further provided herein are methods of preparing an immunostimulatory and/or cytotoxic Pyrularia extract from Pyrularia tissue. In some embodiments, the extracts are immunostimulatory. In other embodiments, the extracts are immunostimulatory and cytotoxic.

The foregoing and other objects, features, and advantages of the invention will become more apparent from the following detailed description, which proceeds with reference to the accompanying figures.

### BRIEF DESCRIPTION OF THE FIGURES

FIG. 1 is a flow cytometry scan that illustrates PyEx-2 treatment simulates murine bone marrow cells. Balb/C bone marrow cells (1.5×10⁶ cells/ml) were cultured alone (FIG. 1A) or in the presence of 2 μg/mL PyEx-2 (FIG. 1B). After 40 hours, 200 μL of cell suspension was stained with anti-Gr-1-FITC and anti-Mac-1-PE and analyzed by means of an EPICS-XL™ flow cytometer as described herein. Those cells staining more intensely along the Y-axis (Mac-1^{+hi}) are activated with PyEx treatment, as seen in FIG. 1B.

FIG. 2 is a graph that shows PyEx reverses cyclophosphamide (CP)-induced white blood cell depletion in Balb/C mice. Blood from Balb/C mice was collected with a heparinized capillary tube from the orbital plexus on the days indicated. Blood was diluted into Isoton II solution (Beckman-Coulter, Fullerton, Calif.). Total cells were estimated by means of an electronic cell counter as described herein. White blood cells were counted after lysis of red cells.

FIG. 3 is a flow cytometry scan showing that PyEx treatment restores Balb/C blood granulocytes and re-colonization of bone marrow, after injection of a sub-lethal dose of CP. The normal abundance of blood granulocytes (FIG. 3A) and bone marrow cells (FIG. 3B) was severely depleted following CP treatment, as shown in FIG. 3C and FIG. 3D, respectively. Treatment with PyEx led to reversal of this immunosuppression, as shown by restoration of granulocytes (FIG. 3E) and recolonization of bone marrow (FIG. 3F). All cell samples were labeled with Gr-1-FITC and Mac-1-PE and analyzed by flow cytometry.

FIG. 4 is a graph showing that PyEx-2 stimulates human bone marrow cells. Fresh marrow from a normal, 40-year old donor were incubated without treatment (FIG. 4A), with PyEx-2 (2 μg/mL; FIG. 4B) or with rhGM-CSF (5 ng/mL; FIG. 4C) for 48 hours prior to analysis by flow cytometry. Cells were labeled with Gr-1-FITC and Mac-1-PE.

FIG. 5 is a graph showing the MALDI mass spectrum of a mixture of oligosaccharides following deamination. The oligosaccharides were obtained from purification of LPS core derived from Pyrularia extracts.

FIG. 6 is a graph showing the reconstructed ESI mass spectrum of O-deacylated LPS obtained from Pyrularia extracts.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Sep. 16, 2010 (˜1.45 KB), which is incorporated by reference herein. In the accompanying sequence listing:

SEQ ID NO: 1 is the nucleotide sequence of a 16S forward PCR primer.

SEQ ID NO: 2 is the nucleotide sequence of a 16S reverse PCR primer.

SEQ ID NO: 3 is the nucleotide sequence of a PCR amplification product of genomic DNA isolated from Pyrularia-associated bacteria.

### DETAILED DESCRIPTION

### I. Abbreviations

BM Bone marrow

### CP Cyclophosphamide

DHB 2,4-dihydroxybenzoic acid
DNA Deoxyribonucleic acid
ESI Electrospray ionization
EU Endotoxin units
FITC Fluorescein isothiocyanate
GC-MS Gas chromatography-mass spectrometry
HMBC Heteronuclear multiple bond correlation

### IFN Interferon

### IL Interleukin

i.p. Intraperitoneal
KDO 2-keto-3-deoxyoctonate
LAL Limulus amoebocyte lysate

### LB Luria-Bertani

### LPS Lipopolysaccharide

LPS-FF Lipopolysaccharide-fruit fraction
LPS-P Lipopolysaccharide-Pantoea ananatis/agglomerans
MALDI Matrix-assisted laser desorption ionization
MS Mass spectrometry
MWCO Molecular weight cut-off
NMR Nuclear magnetic resonance
NO Nitric oxide
PAGE Polyacrylamide gel electrophoresis
PBS Phosphate-buffered saline

### PE Phycoerythrin

PEC Peritoneal exudate cells
PyEx Pyrularia fruit extract
PyEx-1 25% ammonium sulfate-extract of Pyrularia fruit
PyEx-2 Pyrularia extract further purified by anion-exchange chromatography
RBC Red blood cell
rhGM-CSF Recombinant human granulocyte macrophage-colony stimulating factor
RPM Revolutions per minute
SDS Sodium dodecyl sulfate

### SRB Sulforhodamine-B

SPBD Single phase Bligh-Dyer
TNF-αTumor necrosis factor-α
WBC White blood cell

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: The introduction of a composition into a subject by a chosen route. For example, if the chosen route is intravenous, the composition is administered by introducing the composition into a vein of the subject.

Analog: A molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with techniques such as those disclosed in Remington: The Science and Practice of Pharmacology, 19^{th} Edition (1995), chapter 28. A derivative is a biologically active molecule derived from the base structure. A mimetic is a biomolecule that mimics the activity of another biologically active molecule. Biologically active molecules can include both chemical structures and peptides of protein entities that mimic the biological activities of the Pyrularia extracts of the present invention.

Animal: Living multi-cellular vertebrate organisms, a category that includes, for example, mammals and birds. The term mammal includes both human and non-human mammals. Similarly, the term “subject” includes both human and veterinary subjects. Likewise, the term mammal includes both human and non-human mammals.

Biologically active compound: A compound that displays a measurable activity in a biological system is considered to be biologically active (bioactive).

Cancer: A cancer is a biological condition in which a neoplasm has undergone characteristic anaplasia with loss of differentiation, increased rate of growth, in some cases invasion of surrounding tissue, and which is capable of metastasis. The resultant neoplasm is also known as a malignant tumor.

The term cancer includes breast carcinomas (e.g. lobular and duct carcinomas), and other solid tumors, sarcomas, and carcinomas of the lung such as small cell carcinoma, large cell carcinoma, squamous carcinoma, and adenocarcinoma, mesothelioma of the lung, colorectal adenocarcinoma, stomach carcinoma, prostatic adenocarcinoma, ovarian carcinoma such as serous cystadenocarcinoma and mucinous cystadenocarcinoma, ovarian germ cell tumors, testicular carcinomas, and germ cell tumors, pancreatic adenocarcinoma, biliary adenocarcinoma, hepatocellular carcinoma, bladder carcinoma including transitional cell carcinoma, adenocarcinoma, and squamous carcinoma, renal cell adenocarcinoma, endometrial carcinomas including adenocarcinomas and mixed Mullerian tumors (carcinosarcomas), carcinomas of the endocervix, ectocervix, and vagina such as adenocarcinoma and squamous carcinoma, tumors of the skin like squamous cell carcinoma, basal cell carcinoma, melanoma, and skin appendage tumors, esophageal carcinoma, carcinomas of the nasopharynx and oropharynx including squamous carcinoma and adenocarcinomas, salivary gland carcinomas, brain and central nervous system tumors including tumors of glial, neuronal, and meningeal origin, tumors of peripheral nerve, soft tissue sarcomas and sarcomas of bone and cartilage. Also included are non-solid hematopoietic tumors, such as leukemias and lymphomas.

CD11b and Mac-1: CD11b is the α subunit of the heterodimeric integrin Mac-1 (also known as α_{M}β₂ or complement receptor 3). Mac-1, which is comprised of CD11b and CD18, is expressed on monocytes, macrophages, granulocytes and natural killer cells.

Cytokine: A type of protein secreted by cells of the immune system. Cytokines regulate the immune system through specific effects on cell-cell interaction, communication and behavior of other cells.

Cytotoxic: Refers to a substance that is toxic to cells.

Differentiation: Process by which cells become more specialized to perform biological functions. Differentiation is a property that is completely or partially lost by cells that have undergone malignant transformation.

Gr-1: A GPI-linked protein expressed on the surface of mature granulocytes in the bone marrow and on peripheral neutrophils. Gr-1 is also known as Ly6G.

Granulocyte: A white blood cell having a multi-lobed nucleus and cytoplasmic granules. Granulocytes, also known as polymorphonuclear leukocytes, include neutrophils, eosinophils and basophils. In some embodiments herein, granulocytes are characterized by expression of Gr-1.

Immunostimulatory: Refers to the activation or stimulation of any aspect of the immune system. Thus, a compound that causes stimulation of any aspect of the immune system is said to be “immunostimulatory” or to have an “immunostimulatory activity.”

One specific type of immunostimulatory activity is granulocyte-stimulatory activity. This includes any treatment that causes the activation or stimulation of granulocytes, for instance granulocytes derived from peripheral blood or from bone marrow. Activation or stimulation of granulocytes can be measured, for instance, by examining an increase in the number of cells isolated from peripheral blood or bone marrow after a treatment compared with the number isolated from a control (e.g., untreated or pre-treated) sample, or as seen by more intense staining with markers (i.e., fluorescent-labeled monoclonal antibodies) specific to this cell type (for example, Gr-1). A compound or composition that causes such granulocyte stimulation, or is capable of activating granulocytes, is said to have a granulocyte-stimulatory activity. Similarly, a compound or composition that is capable of activating macrophages is said to have macrophage-stimulating activity.

Incorporation of biologically active compound into pharmaceutical compositions: Pharmaceutical compositions that comprise at least one biologically active compound (e.g., a lipopolysaccharide) as described herein as an active ingredient will normally be formulated with an appropriate solid or liquid carrier, depending upon the particular mode of administration chosen. The pharmaceutically acceptable carriers and excipients useful in this invention are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate.

Other medicinal and pharmaceutical agents, for instance immunostimulatory compounds, also may be included.

The dosage form of the pharmaceutical composition will be determined by the mode of administration chosen. For instance, in addition to injectable fluids, topical and oral formulations can be employed. Topical preparations can include eye drops, ointments, sprays and the like. Oral formulations may be liquid (e.g., syrups, solutions or suspensions), or solid (e.g., powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those skilled in the art.

The pharmaceutical compositions that comprise biologically active compounds as described herein (e.g., mitogenic and/or cytotoxic plant extracts, or biologically active lipopolysaccharide) may be formulated in unit dosage form, suitable for individual administration of precise dosages. One possible unit dosage contains approximately 3 mg/m² of active extract (or preparation) containing lipopolysaccharide. The amount of active compound administered will be dependent on the subject being treated, the severity of the affliction, and the manner of administration, and is best left to the judgment of the prescribing clinician. Within these bounds, the formulation to be administered will contain a quantity of the active component(s) in an amount effective to achieve the desired effect in the subject being treated.

Injectable composition: A pharmaceutically acceptable fluid composition comprising at least one active ingredient, e.g., a mitogenic active component of a Pyrularia fruit extract. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions for use with the mitogenic and/or cytogenic extracts and compounds of this invention are conventional; appropriate formulations are well known in the art (see, for example U.S. Pat. No. 5,609,868).

Isolated: An “isolated” biological component (such as a nucleic acid molecule, lipid, protein or organelle) has been substantially separated or purified away from other biological components in the cell of the organism in which the component naturally occurs, for example other proteins in a naturally occurring mixture, or other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Lipopolysaccharide (LPS): A large molecule consisting of a lipid and a polysaccharide joined by a covalent bond. LPS is found in the outer membrane of Gram-negative bacteria, and elicits strong immune responses in animals. LPS is also referred to as endotoxin.

Macrophage: A cell that originates from specific white blood cells called monocytes. Monocytes and macrophages are phagocytes, acting in nonspecific defense (or innate immunity) as well as specific defense (or cell-mediated immunity) of vertebrate animals. Their role is to phagocytize (engulf and then digest) cellular debris and pathogens either as stationary or mobile cells, and to stimulate lymphocytes and other immune cells to respond to the pathogen. Macrophages are derived from bone marrow precursor cells and are found in most tissues of the body.

Mimetic: A biological compound (such as a peptide) that mimics the effect of a pharmaceutical, for example a peptide that mimics the effect of a biologically active component of a mitogenic and/or cytogenic Pyrularia extract by stimulating the immune system. Particularly, the activity of such mimetic compound(s) can readily be tested by one or more of the mitogen activity assays described herein, or by other such assays known in the art.

Myelocyte: A type of cell typically found in the bone marrow that gives rise granulocytes and macrophages.

Neoplasm: A new and abnormal growth, particularly a new growth of tissue or cells in which the growth is uncontrolled and progressive. A tumor is an example of a neoplasm.

Non-native sequence or structure: As used herein, a compound having a non-native sequence or structure refers to the modification to a natural compound, e.g., a protein, glycoprotein, or oligo- or polysaccharide, lipid, phospholipid, glycolipid, or lipopolysaccharide. In certain embodiments, compounds that have a non-native sequence or structure maintain the mitogenic and/or cytotoxic activities described herein. Activity-preserved, non-native saccharide chains, whether independent of a protein (e.g., oligo- or polysaccharides) or attached to a protein (e.g., in a glycoprotein) may contain one or more saccharide (e.g., monosaccharide) substitutions, deletions or additions. Likewise, activity-preserved proteins may contain one or more deletions, additions, or conservative amino acid substitutions.

Pantoea: A genus of Gram-negative bacteria of the family Enterobacteriaceae. The Pantoea genus includes seven species (P. ananatis, P. agglomerans, P. citrea, P. dispersa, P. punctata, P. stewartii and P. terrea). Pantoea species are usually isolated from soil, fruit and vegetables. As used herein, a Pantoea species “associated with” Pyrularia fruit is any Pantoea species that grows, or is isolated from, the surface of Pyrularia fruit. In some embodiments, the Pantoea species is P. ananatis or P. agglomerans.

Parenteral: Administered outside of the intestine, e.g., not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance. Parenteral administration is usually preferred for peptide drugs, to avoid gastric degradation of the peptide.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of therapeutic agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent or drug: A chemical compound or composition capable of inducing a desired therapeutic or prophylactic effect when properly administered to a subject.

Purification of bioactivity: Compounds as described herein can be purified by any of the means known in the art, including the specifically described methods herein. See, e.g., Guide to Protein Purification, ed. Deutscher, Meth. Enzymol. 185, Academic Press, San Diego, 1990; and Scopes, Protein Purification: Principles and Practice, Springer Verlag, New York, 1982. For isolation of lipopolysaccharides, see McIntire et al., Biochemistry 6:2363-2372, 1967; Manthey and Vobel, J. Endotoxin Res. 1:84-91, 1994; Hirschfield et al., J. Immunol. 165:618-622, 2000; and Yi and Hackett, Analyst 125:651-656, 2000.

Purified, homogeneous compounds: The term purified does not require absolute purity; rather, it is intended as a relative term. Thus, for example, a purified preparation is one in which the desired compound (e.g., a lipopolysaccharide) is more enriched than it is in its natural environment (within a cell or a laboratory production vessel). A compound (e.g., a biologically active compound of a Pyrularia extract as described herein) is “purified” if it has been substantially separated from contaminants, e.g., cellular components (nucleic acids, lipids, carbohydrates, and other polypeptides) that naturally accompany it. Such a compound can also be referred to as “pure” or “homogeneous” or “substantially” pure or homogeneous. Purified is intended to be a relative term, and a compound is purified when at least 50-90% by weight of a sample is composed of the compound. In certain highly purified preparations, the subject compound will be at least 95% or more of the sample, even as much as 99% or more. Purity or homogeneity is indicated, for example, by polyacrylamide gel electrophoresis; high pressure liquid chromatography; or other conventional methods.

Pyrularia extract: A concentrated preparation of Pyrularia pubera fruit or root that contains immunostimulatory activity.

Pyrularia pubera: A terrestrial parasitic plant that grows on the roots of various broadleaf tree and shrubs (such as oak trees). Pyrularia pubera is commonly referred to as “buffalo nut.”

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Similarly, a recombinant protein is one encoded by a recombinant nucleic acid molecule.

Subject: Living multi-cellular vertebrate organisms, a category that includes both human and veterinary subjects, including human and non-human mammals.

Therapeutic agent: Used in a generic sense, it includes treating agents, prophylactic agents, and replacement agents.

Therapeutically effective amount: A quantity of a specified compound or extract preparation sufficient to achieve a desired effect in a subject being treated. For instance, this can be the amount of a Pyrularia fruit extract (or a biologically active component thereof) necessary to stimulate an immune cell population in a subject (e.g., to stimulate myelocytes in a subject). Specific examples would be a therapeutically effective amount of a Pyrularia fruit extract (e.g., PyEx-1 or PyEx-2), or a mitogenic active component thereof (e.g., a lipopolysaccharide purified from a Pyrularia fruit extract, and having mitogenic activity). For instance, this can be the amount of Pyrularia fruit extract necessary to stimulate the immune system of a subject (also referred to as an immunostimulatory effective amount of the extract). Specific immunostimulatory effects that can be caused by Pyrularia fruit extracts (as exemplified by PyEx-1 and PyEx-2), and by mitogenic active components of such extracts, are described herein. Ideally, an immunostimulatory amount of an extract or purified component is an amount sufficient to stimulate an immune response (for instance, any of the stimulatory responses discussed herein) without causing a substantial cytotoxic effect (e.g., without killing more than 10% of cells in a sample). However, the effective amount of Pyrularia fruit extract, or active component thereof, will be dependent on the subject being treated, the severity of the affliction, and the manner of administration of the therapeutic composition

An effective amount of an immunostimulatory compound may be administered in a single dose, or in several doses, for example daily, during a course of treatment. However, the effective amount of the compound will be dependent on the preparation applied, the subject being treated, the severity and type of the affliction, and the manner of administration of the compound. For example, a therapeutically effective amount of Pyrularia extract can vary from about 0.01 mg/kg body weight to about 1 g/kg body weight in some embodiments, or from about 0.01 mg/kg to about 60 mg/kg of body weight, based on efficacy and potential toxicity.

The mitogenic and/or cytotoxic Pyrularia extracts disclosed in the present invention (and isolated biologically active components thereof) have equal applications in medical and veterinary settings. Therefore, the general terms “subject” and “subject being treated” are understood to include all animals, including humans or other simians, dogs, cats, horses, and cows.

Tumor: A neoplasm that may be either malignant or non-malignant and includes both solid and non-solid tumors (such as hematologic malignancies). “Tumors of the same tissue type” refers to primary tumors originating in a particular organ (such as breast, prostate, bladder, or lung). Tumors of the same tissue type may be divided into tumor of different sub-types (a classic example being bronchogenic carcinomas (lung tumors) which can be an adenocarcinoma, small cell, squamous cell, or large cell tumor). Breast cancers can be divided histologically into scirrhous, infiltrative, papillary, ductal, medullary, and lobular. As used herein, “inhibiting tumor development or growth” refers to slowing or preventing the initiation of a tumor, or decreasing the rate of growth or size of the tumor. “Inhibiting tumor metastasis” includes preventing metastasis, slowing the progression of metastasis or delaying the formation of metastases.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Disclosed herein is the finding that Pyrularia plants harbor epiphytic and/or endophytic bacteria, including Pantoea agglomerans or Pantoea ananatis. It is further disclosed herein that LPS isolated from the Pyrularia plant, or LPS isolated from the plant-associated bacteria (such as Pantoea agglomerans or Pantoea ananatis), possesses potent immunostimulatory activity. This immunostimulation activity increases the number of murine or human granulocytes, promotes restoration of white blood cell counts, and decreases the time required for bone marrow re-colonization and restoration of blood granulocytes following sub-lethal chemotherapy.

Thus, provided herein are immunostimulatory compositions comprising LPS isolated from Pyrularia fruit extracts. Also provided are immunostimulatory compositions comprising LPS isolated from a Pantoea species associated with Pyrularia fruit. In some embodiments, the LPS is isolated from Pantoea ananatis or Pantoea agglomerans. The immunostimulatory compositions disclosed herein are capable of activating granulocytes and/or macrophages.

In some embodiments, the LPS is substantially stable at 100° C. and/or the LPS is stable at a pH of 3-10 at 25° C.

In some embodiments disclosed herein, the compositions further comprise a pharmaceutically acceptable carrier.

Further provided is a method of stimulating immunity by administering to a subject in need thereof an immunostimulatory composition disclosed herein. In some embodiments, the method further includes administering to the subject a therapeutically effective amount of a second immunostimulatory composition. In some examples, the second immunostimulatory composition comprises a cytokine.

In some embodiments of the methods, stimulating immunity comprises inhibiting tumor development or growth in the subject, and the method comprises administering the agent to a subject having, or at risk of developing, a tumor. In some examples, inhibiting tumor development comprises inhibiting tumor metastasis.

In some embodiments, stimulating immunity comprises activating granulocytes and/or macrophages. In some embodiments, stimulating immunity comprises inducing mitosis in an immune cell in the subject.

Also provided is a method of preparing an immunostimulatory and/or cytotoxic Pyrularia extract. In some embodiments, the method includes extracting Pyrularia tissue in a fluid to form a primary extract; precipitating the primary extract with 15% ammonium sulfate to form a secondary extract; and dialyzing the secondary extract using 12-14 kD MW cutoff dialysis tubing to produce the immunostimulatory and/or cytotoxic Pyrularia extract. As disclosed herein, Pyrularia plants harbor bacteria, including Pantoea agglomerans or Pantoea ananatis, which provide the immunostimulatory and/or cytotoxic component of the extract.

In some embodiments, the method further includes producing a more highly purified Pyrularia extract by passing the immunostimulatory and/or cytotoxic Pyrularia extract over a size exclusion column; and collecting high molecular weight fractions to yield the more highly purified Pyrularia extract.

Further provided is a method of preparing an immunostimulatory Pyrularia extract. In some embodiments, the method includes passing the more highly purified Pyrularia extract disclosed herein over an anion exchange column; and collecting low salt (for example, 10 mM ammonium carbonate) eluate from the column, to form the immunostimulatory Pyrularia extract. In some examples, the method further includes heating the immunostimulatory Pyrularia extract to at least 60° C. for at least 10 minutes.

Also provided is a method of preparing a cytotoxic Pyrularia extract that includes passing the more highly purified Pyrularia extract disclosed herein over an anion exchange column; and collecting high salt (for example, 1M NaCl) eluate from the column, to form the cytotoxic Pyrularia extract.

In some embodiments of the methods of preparing an immunostimulatory and/or cytotoxic Pyrularia extract, the immunostimulatory and/or cytotoxic Pyrularia extract comprises LPS from a bacterium associated with the Pyrularia tissue. In other embodiments, the immunostimulatory and/or cytotoxic Pyrularia extract comprises LPS from a Pantoea bacterium associated with the Pyrularia tissue. In particular examples, the Pantoea bacterium associated with the Pyrularia tissue is Pantoea agglomerans or Pantoea ananatis.

Further provided is a Pyrularia extract with immunostimulatory and/or cytotoxic activity, prepared by any one of the methods disclosed herein. In some embodiments, the extract has granulocyte-stimulatory activity.

### IV. Pyrularia Fruit Extracts

A. Granulocyte-Stimulatory Activity of Pyrularia pubera Extracts

A natural product (PyEx) derived from the plant Pyrularia pubera demonstrated in vitro immunostimulatory activity toward murine bone marrow cells. PyEx prevented drug-associated toxic death in Balb/C mice. In addition, treatment with PyEx led to a hastened recovery of total white blood cells and restoration of total blood granulocytes to 130% of that found in saline-treated control mice five days following sub-lethal treatment with cyclophosphamide (CP), compared to less than 1% in the CP alone group.

Flow cytometric analysis of murine bone marrow treated with PyEx revealed an activated sub-population which stained positively with either anti-Mac-1(CD11b)FITC×anti-Mac-1-PE or with anti-Gr-1-FITC×anti-Mac-1-PE. This is indicative of stimulation of granulopoiesis.

Similar activation was demonstrated with human bone marrow cells by labeling with anti-Mac-1-FITC×anti-CD14-PE, indicating that this plant-derived natural product is also useful in the treatment of certain human immune deficient conditions, such as drug-induced myelosuppression.

B. Extraction Procedures

In one particular extraction procedure, a crude fraction of the Pyrularia pubera fruit is obtained from a basic fraction (see Example 2), such as ammonium sulfate extraction. This crude fraction was found to have both cytotoxic and mitogenic (immunostimulatory) activity. These combined activities are particularly useful in the treatment of tumors, because the cytotoxic effect can directly damage the rapidly dividing tumor cells while the immunostimulatory effect offsets toxic effects on hematopoiesis. Immune stimulation can also recruit the subject's immune defenses against the tumor.

Further purification of the crude fraction, for example on an anion exchange column, yields a more highly purified fraction (PyEx-2) that is not cytotoxic, but which has mitogenic (immunostimulatory) activity. This activity is stable when the PyEx-2 fraction is heated to >80° C., and is stable across a range of pH (3-10 at 25° C.). The activity also is resistant to treatment with proteolytic enzymes, indicating that the active compound is not a common protein.

The immunostimulatory but not cytotoxic purified fraction is particularly suited for administration as an immunostimulant, for example to an immuno-compromised individual who does not have a tumor, such as a leukopenic individual, for example someone who is granulopenic. The cytotoxic component can be inactivated by heating Pyrularia extract at 70° C. for 20 minutes.

LPS can also be directly isolated from Pyrularia-associated bacteria, such as by using the solvent extraction procedure as described in Example 13. LPS obtained from Pyrularia-associated bacteria is immunostimulatory.

A number of methods of isolating LPS from bacteria are known in the art and can be applied to extract immunostimulatory LPS from Pyrularia-associated bacteria. For example, the LPS isolation procedure can include phenol-water extraction of bacteria (e.g. phenol-water extraction of a bacterial cell paste; see Example 13 below) and/or low or high speed centrifugation to remove debris or separate bacterial cell components. Additional steps can be taken to further purify crude LPS preparations obtained from bacterial samples, such as nuclease treatment (e.g., RNAse or DNAse treatment), size-exclusion chromatography, anion-exchange chromatography, and/or FPLC anion-exchange chromatography.

C. Activities

PyEx activity was initially identified in a screen for immunologically active materials. It was found that PyEx possesses mitogenic activity toward mouse splenocytes and possesses synergistic activity in combination with certain other mitogenic agents. PyEx also demonstrated a mitogenic response in mouse bone marrow cells. In particular, it was found by flow cytometry to stimulate a subset of these cells that are double positive for labeling with Gr-1 and Mac-1 monoclonal antibodies (Gr-1⁺, Mac-1⁺), which is indicative of activation of a myelocytic cell lineage in the bone marrow and mature granulocytes (specifically neutrophils) in peripheral blood (Fine et al., Blood, 90:795, 1997).

Using a mouse neutropenia model (Hattori et al., Blood, 75:1228, 1990), it was found that PyEx induced granulocytosis in vivo in Balb/C mice and accelerated the recovery of leukocytes, and more specifically neutrophils, following chemotherapy. Moreover, PyEx treatment prevented drug-induced lethality in mice. This pharmacological profile is remarkably similar to that demonstrated by G-CSF and GM-CSF (Tamura et al., Biochem. Biophys. Res. Commun. 142:454, 1987; Cohen et al., Proc. Natl. Acad. Sci. USA 84:2484, 1987).

In addition, the immunostimulatory activity of Pyrularia fruit extracts is demonstrated in vitro with fresh bone marrow cells, suggesting that this effect is directed toward bone marrow rather than an induced immunomodulation or a separate immunological response. Indeed, bone marrow stimulation is evident under conditions where mitogenic and PEC-stimulation is inhibited. Unlike CSF-like compounds currently under examination by other groups, which appear to be effective in mice without showing activity in human cells in vitro (Tian et al., Anticancer Res., 19:237, 1999), Pyrularia extract has strong demonstrable activity in human bone marrow cultures.

LPS isolated from purified bacteria obtained from Pyrularia fruit is also shown to have immunostimulatory activity. As shown for Pyrularia fruit extracts, LPS from Pyrularia-associated bacteria is capable of activating bone marrow cells.

In summary, in some embodiments Pyrularia extracts described herein, and the mitogenic components thereof, are potent compositions for treatment of neutropenia in clinical settings.

### V. Active Components of Pyrularia Extract

Bone marrow stimulating activity could be extracted from Pyrularia fruit extracts with boiling MeOH. However, little comparable activity was able to be extracted directly from ground Pyrularia root or from fruit prepared in the presence of antibiotics or with incubation at 4° C., suggesting the possibility that in spite of surface sterilization, bacterial products are present in the Pyrularia extracts.

A. Identification of Phytopathogenic Bacteria Pantoea ananatis

As described in Example 16, the bacteria present in Pyrularia fruit extracts was identified as Pantoea ananatis. Bacteria from Pyrularia extracts were grown on LB plates to isolate single colonies. Isolated bacterial colonies were expanded in LB broth and bacterial genomic DNA was isolated. Genomic DNA was PCR amplified using primers specific for 16S ribosomal nucleic acid. From two individual colonies, the identical nucleotide sequence was obtained. Sequence analysis of the PCR-amplified nucleic acid sequence identified the Pyrularia-associated bacteria as Pantoea ananatis. It is noted, however, that the 16S ribosomal sequence is the same for Pantoea agglomerans, and that subsequent analysis (using a Biolog plate test (database searched: MicroLog; biolog.com) of the cells of ATCC Deposit No. PTA-10285) indicated that the same strain of bacteria originally identified as Pantoea ananatis was Pantoea agglomerans with 94% probability. It is not believed that the culture is mixed or contaminated; instead, it is proposed that these species may be different in name alone.

B. Isolation of Pyrularia-Associated-Bacterial LPS

Tests for the involvement of bacterial products were conducted, and it was found that lipopolysaccharide (LPS) isolated from the Pyrularia-associated bacteria was involved in the activity of Pyrularia extracts as shown in Examples 8, 12 and 14. LPS was extracted from PyEx or from Pyrularia-associated bacteria directly as described in the Examples below, and/or by one or several procedures described in one of the following references:

1. McIntire et al., Biochemistry 6:2363-2372, 1967

2. Manthey and Vobel, J. Endotoxin Res. 1:84-91, 1994

3. Hirschfield et al., J. Immunol. 165:618-622, 2000

4. Yi and Hackett, Analyst 125:651-656, 2000.

### VI. Incorporation into Pharmaceutical Compositions

Pharmaceutical compositions that include at least one Pyrularia extract (e.g., PyEx-1 or PyEx-2), or a biologically active component thereof (e.g., a lipopolysaccharide) as described herein as an active ingredient, or that include both one of these compounds/compositions and an additional immunostimulatory factor (e.g., an immunostimulatory cytokine) as active ingredients, may be formulated with an appropriate solid or liquid carrier, depending upon the particular mode of administration chosen. The pharmaceutically acceptable carriers and excipients useful in this invention are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate.

The dosage form of the pharmaceutical composition will be determined by the mode of administration chosen. For instance, in addition to injectable fluids, topical and oral formulations can be employed. Topical preparations can include eye drops, ointments, sprays and the like. Oral formulations may be liquid (e.g., syrups, solutions, or suspensions), or solid (e.g., powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art.

The pharmaceutical compositions that comprise a Pyrularia fruit extract (such as PyEx-1 or PyEx-2), or a biologically active component thereof, in some embodiments of the invention will be formulated in unit dosage form, suitable for individual administration of precise dosages. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

### VII. Therapeutic Uses

Doses of immunostimulatory Pyrularia extract (such as PyEx-1 or PyEx-2), or mitogenic components thereof, are effective for treatment of conditions or diseases that affect the immune system, for instance conditions (including clinical treatments) that inhibit (or suppress) the immune system. The immunostimulatory effect can be enhanced by co-administering one or more additional immunostimulants (such as IL-2 or GM-CSF) with the extract or active component. General information about the therapeutic use of immunomodulatory compounds is well known, and can be found for instance in U.S. Pat. Nos. 5,632,983; 5,726,156; and 5,861,483.

In addition, it is contemplated herein to use the immunostimulatory compositions disclosed herein as a feed supplements, such as for poultry or livestock.

A. Susceptible Diseases/Conditions

Immune deficiencies (e.g., deficiencies of one or more immune cells, or of one or more immunological factors) associated with immune deficiency diseases, immune suppressive medical treatment, acute and/or chronic infection, and aging can be treated using the methods and compositions described herein. A general overview of immunosuppressive conditions and diseases can be found in Harrisons “Principles of Internal Medicine,” 14^{th} Edition, McGraw-Hill, 1998, and particularly in chapter 86 (Principles of Cancer Therapy), chapter 88 (Melanoma and other Skin Cancers), chapter 307 (Primary Immune Deficiency Diseases), and chapter 308 (Human Immunodeficiency Virus Diseases).

Immune Suppressive Medical Treatment: Many medical treatments can impair the immune system. Corticosteroids, for example, can reduce cell-mediated immunity. The predominant toxicity associated with cancer therapies (e.g., chemotherapy and radiotherapy) is destruction of proliferating cells, such as hematopoietic cells, responsible for maintenance of the immune and blood systems. Likewise, immune suppression and depletion of the immune system is required for bone marrow transplantation, in which all hematopoietic cells are eliminated and subsequently replaced with transplanted cells. Certain known bone marrow stimulants (e.g., erythropoietin and colony stimulating factors such as GM-CSF or G-CSF, which is sometimes marketed under the name “Neupogen,” U.S. Pat. No. 5,536,495) have been used previously to treat certain of these conditions by stimulating hematopoiesis, for example the production of immune cells such as leukocytes (e.g., lymphocytes, monocytes and macrophages). The immunostimulatory compounds and mixtures of the invention can be used to stimulate the immune systems of patients suffering from medical treatment or iatrogenically induced immune suppression, including those who have undergone bone marrow transplants, chemotherapy, and/or radiotherapy.

Acute and/or Chronic Infection: The ability of Pyrularia fruit extracts, and the immunostimulatory components thereof, to facilitate splenic T cell generation indicates that these extracts and components could be used as a general immunostimulant to activate the immune system against various diseases, both chronic and acute. Subject infections include bacterial and viral infections, as well as infestations caused by eukaryotic pathogens and parasites.

More particularly, immunostimulatory PyEx treatment can be used in the treatment of HIV infection. The ability of extracts and components of Pyrularia fruit extracts, extracted and purified as described herein, to stimulate macrophages and cytotoxic T cells will be beneficial in helping to destroy HIV-infected cells.

Age-Linked Immune Deficiency: Activation of the immune system (via stimulation of T cell production) by PyEx treatment (with or without an added immunostimulant) is also believed to be beneficial in aging subjects, in whom immune function is often compromised.

Other Conditions: In addition to immunosuppression caused by medical treatment and infection, the disclosed methods and compositions can be used to treat genetic and biochemical immune deficiencies.

Several conditions are known in which the immune system is compromised or suppressed, but which do not involve immunosuppressive drug treatment, aging, or disease. Any of these further conditions would also benefit from the methods disclosed herein, or application of the described compositions. In general, the need for treatment with one of the methods or compositions of this invention can be determined by examining the immune status of a test subject, and comparing this immune status to a control or average immune state (a hypothetical “normal” subject). For example, bone marrow biopsies or peripheral blood lymphocytes can be sampled to assess immune function. Indications of reduced immune function include leukopenia, for example neutropenia or lymphopenia, or evidence of diminished white blood cell function. Where the test subject has a reduced immune status, such as a reduction in a peripheral white blood cell count to below normal, for example 25% below normal, the immunostimulatory methods and/or compositions of the invention are available as treatments for the immune suppressed condition.

B. Mode of Treatment

The Pyrularia extracts and purified components thereof (e.g., lipopolysaccharides) may be administered to humans, or other animals on whose cells they are effective, in various manners such as topically, orally, intravenously, intramuscularly, intraperitoneally, intranasally, intradermally, intrathecally, and subcutaneously. The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

Site-specific administration of the disclosed compounds may be used, for instance by applying a Pyrularia preparation (or an immunostimulatory active component thereof) to a pre-cancerous region, a region of tissue from which a neoplasm has been removed, or a region suspected of being prone to neoplastic development.

C. Combinations

The present invention also includes combinations of a Pyrularia extract (e.g., PyEx-1 or PyEx-2), or a purified bioactive component thereof, with one or more other agents useful in the treatment of an immune-related disorder, condition, or disease, e.g. immune suppression caused by a disease or a therapeutic treatment. For example, the extracts and/or extract components of this invention may be administered in combination with effective doses of other immunostimulants, anti-cancer agents, anti-inflammatories, anti-infectives, and/or vaccines. The term “administration in combination” or “co-administration” refers to both concurrent and sequential administration of the active agents.

Co-administration of a Pyrularia extract (or purified active component thereof) with immunostimulants in vaccines is also useful in prevention and/or protection against diseases that affect the immune system.

Other medicinal and pharmaceutical agents, for instance other immunostimulants or agents to prevent certain side effects, may be included. Immunostimulants include levamisole, isoprinosine, muramyl dipeptide, colony stimulating factors such as G-CSF, M-CSF, and GM-CSF, IL-1, IL-2, and other agents. Effective doses of some of these immunostimulatory agents are provided in Munson, Principles of Pharmacology, 1995, pages 1151-1157. Agents may be used in combination with PyEx to eliminate certain undesirable side effects. These include for example, methyl xanthines (e.g., pentoxyifyilline), rolipram, quercetin phosphodiesterase inhibitors (e.g., roflumilast), or agents that elevate cAMP levels or stimulate various cellular protein kinases. In addition, agents 1) which are known to prevent symptoms leading to septic shock such as a) inhibitors of IL-1β converting enzyme (ICE) or b) IL-Ira (interleukin1 receptor antagonist), 2) anti-inflammatory cytokines such as IL-10, IL-4, or IL-13, or 3) agents which inhibit the production or action of pro-inflammatory cytokines, including IL-1, IL-6, TNFα, NO, or IFN may be used in combination chemotherapy.

The combination therapies are of course not limited to the lists provided in these examples, but include any composition for the treatment of an immune disorder, such as an immunosuppressive disorder.

D. Timing of Treatment

Treatment of a subject using the immunostimulatory compositions of the invention may be indicated after (or while) the subject has received an anti-proliferative or other cytotoxic therapeutic treatment. Examples of anti-proliferatives compounds include the following: ifosamide, cisplatin, methotrexate, cytoxan, procarizine, etoposide, BCNU, vincristine, vinblastine, cyclophosphamide, taxol, gemcitabine, 5-fluorouraci, paclitaxel, and doxorubicin.

In some embodiments of the invention, a subject is given a cytotoxic treatment, then monitored for a period of time (usually in the range of days to weeks) to determine if the treatment leads to an immunosuppressive effect. Such monitoring can include monitoring peripheral blood for leukopenia or pancytopenia, and/or monitoring T cell function. A subject that displays an immune suppression will be a candidate for treatment using the compounds (e.g., a Pyrularia extract such as PyEx-2, or a purified immunostimulatory component of such extract) and therapeutic methods of the disclosed invention.

Though both PyEx-1 and PyEx-2 have immunostimulatory activity, there are instances in which it would be more appropriate to administer one or the other of the extracts, or more particularly purified active components thereof. For instance, the PyEx-2 extract of the invention, having immunostimulatory activity, is beneficially administered to subjects in need of stimulation of their immune system, for instance subjects who are immune suppressed as described herein. PyEx-1 can be administered for the treatment of hyperproliferative conditions, such as tumors, where both cytotoxic and immunostimulatory effects are desired.

### VIII. Kits

The Pyrularia extracts, for instance PyEx-1 and PyEx-2, and purified active components thereof, can be supplied in kits for use in stimulation of an immune system, or for the prevention and/or treatment of a disorder, condition or diseases (e.g., an immune-compromised condition). Alternatively, PyEx-1 can be included in a kit for the treatment of hyper-proliferative conditions, such as tumors, where both cytotoxic and immunostimulatory effects are desired. In kits provided herein, a clinically effective amount of one or more of the extracts or bioactive components (e.g., an effective amount of PyEx-2, or a purified immunostimulatory active component thereof) is provided in one or more containers. The compositions may be provided suspended in an aqueous solution or as a freeze-dried or lyophilized powder, for instance. In certain embodiments, the compositions will be provided in the form of a pharmaceutical composition.

Kits according to this invention can also include instructions, usually written instructions, to assist the user in treating a disorder, condition, or disease (e.g., a tumor or an immunodeficiency) with a Pyrularia extract, such as PyEx-1 and PyEx-2, or a purified active component thereof. Such instructions can optionally be provided in non-printed format, such as in a computer readable medium.

The container(s) in which the composition(s) are supplied can be any conventional container that is capable of holding the supplied form, for instance, microfuge tubes, ampoules, or bottles. In some applications, the therapeutic composition may be provided in pre-measured single use amounts in individual, typically disposable, tubes or equivalent containers.

The total amount of an active extract or compound or combination supplied in the kit can be any appropriate amount, depending for instance on the market to which the product is directed. For instance, if the kit is adapted for research or clinical use, the amount of each Pyrularia extract, such as PyEx-1 or PyEx-2, or a purified active component thereof, provided in a kit would likely be an amount sufficient for several treatments.

Certain kits according to this invention will also include one or more other agents useful in stimulating the immune system, or in inhibiting a tumor, e.g. in treating hyper-proliferation. For example, such kits may include one or more effective doses of other anti-proliferative or anti-cancer drugs, or an immunostimulant such as GM-CSF or an anti-inflammatory.

The following examples are provided to illustrate certain particular features and/or embodiments. These examples should not be construed to limit the disclosure to the particular features or embodiments described.

### EXAMPLE

### Example 1

### General Materials and Assays

This example provides details of materials and methods used throughout the remaining Examples. Unless otherwise stated in a specific example, general materials and methods were as follows:

### Materials

Fluorescent-labeled and unlabeled antibodies were obtained from Pharmingen (San Diego, Calif.). Cyclophosphamide, melphalan and colony stimulating factors, including recombinant human granulocyte macrophage-colony stimulating factor (rhGM-CSF), were obtained from Sigma Chemical Company (St. Louis, Mo.). Isoton II and Zap-Oglobin were obtained from Beckman-Coulter (Fullerton, Calif.). Red cell lysis buffer consisted of 150 mM NH₄Cl, 1 mM KHCO₃, and 1μM Na₂EDTA.2H₂O, pH 7.2. RPMI medium was composed of RPMI-1640 medium (Sigma Chemical Company) supplemented with 10% heat inactivated fetal calf serum and gentamycin.

### Mice

Balb/C or C57B1 mice were obtained from either Charles River (Willmington, Mass.), or Jackson Laboratories (Bar Harbor, Me.). Before experimentation, mice were acclimated for a period of two weeks in a temperature-controlled environment with alternating light/dark cycles of 12 hours and were allowed food and water ad libitum.

### Harvesting of Immunological Cells

Fresh cells were teased from mouse spleens or washed from mouse femurs into RPMI medium. Cells and medium were then transferred to a 15 mL tube and the cells collected by centrifugation at 2000-3000×g for ten minutes. The supernatant media was aspirated and the cells treated with 2 mL red cell lysis buffer for each spleen or 1 mL per two femurs. After five minutes at room temperature, the cells were washed twice in 12 mL of RPMI, cells were pelleted at 2000-3000×g for ten minutes after each wash, and the wash medium removed and discarded. Peritoneal exudate cells (PEC) were collected by peritoneal lavage four days following i.p. injection of 3 ml of 3% thioglycollate medium into Balb/C mice and washed with growth medium. Non-adherent cells were washed from 96-well plates after a two-hour incubation at 37° C.

### Mitogen Assay Using Spleen or Bone Marrow Cells

A mitogenic assay was used as a measure of immunostimulation. For the mitogen assay, isolated cells were diluted to 1.5×10⁶ cells/mL. Aliquots of cells (200 μL/well) were incubated in sterile 96-well plates for 24 to 48 hours prior to the addition of 1 μCi/well of [³H]thymidine. Radiolabeled DNA was collected on glass fiber filters by means of an Inotech cell harvester (Rockville, Md.) and associated radioactivity was determined by means of an Inotech radiometric instrument or by scintillation counting of individual filters. TLF-D, a proprietary reagent, was utilized at 10 μg/ml to amplify the mitogenic activity of Pyrularia extract (PyEx) in a synergistic fashion.

### Bone Marrow Activation Assay

Bone marrow was flushed from the femurs of Balb/C mice with RPMI medium. Cells were collected by centrifugation and the pellet treated with 1 mL of red cell lysis buffer (as described above) for five minutes at 25° C., followed by dilution with 12 mL of RPMI media. Cells were washed by two cycles of centrifugation and resuspension in RPMI. On the final wash, cells were counted using a hemocytometer and the final pellet was resuspended to 4×10⁶ cells/mL with RPMI media for use in activation assays, which were quantitated by flow cytometric analysis (described below).

### Cytotoxicity Assays

Cytotoxic activity toward adherent cells was determined using the sulforhodamine-B (SRB) method of Skehan and coworkers (J. Nat. Cancer Inst. 82: 1107-12, 1990). Briefly, cells were suspended in RPMI-1640 media and placed into a 96 well-plate (1.5×10³ cells/well) in the presence of test cytotoxic extracts or known cytotoxic compounds. After a period of log-phase growth of 18-24 hours, the media was aspirated and protein fixed to the plate by the addition of 75 μl of 0.4 N perchloric acid.

After standing for 15 minutes, the contents in each well were gently rinsed four times with distilled water. Protein in each well was stained by treatment with 100 μl of 4% SRB in 1% acetic acid for 30 minutes. The SRB was then aspirated and the wells were rinsed 4 times with 1% acetic acid. Plates were allowed to dry overnight. Bound SRB in each well was solubilized with 75 μl of 10 mM Tris base (unbuffered). The amount of dye bound was determined spectrophotometrically at 570 nm and was linearly proportional to the amount of protein contained in the well.

### Flow Cytometric Analysis

Cells (200 μL, prepared as described above) were collected after gentle trituration to resuspend cells. Each aliquot was diluted with 1 mL PBS, centrifuged to pellet the cells, and the supernatant liquid was removed. The washed cells were sequentially stained in the dark at 4° C. for 15 minutes with anti-CD16/32 to prevent non-specific labeling, then for 45 minutes with either anti-Mac-1-FITC×anti-Mac-1-PE or with Gr-1-FITC×Mac-1-PE. Cells were washed once at 4° C. with 1 mL of PBS and then resuspended in 1 mL of PBS. Human cells were labeled with anti-Mac-1 as above or with anti-Mac-1-FITC×anti-CD14. A Coulter EPICS-XL™ flow cytometer was used to enumerate dual, positively-staining control sub-populations and detection of nascent stimulated sub-populations. Activated sub-populations, which labeled with more intensity along the y-axis in Gr-I-FITC×Mac-1-PE, were designated as Mac-1^{+hi}. Control unactivated cells that also stained positively in both dimensions were designated, Mac-1^{+lo}. Dead cells were eliminated from analysis by prior staining with propidium iodide. Approximately 15-20×10³ cells were counted for each analysis. Percent of cells activated was determined as:

$$\frac{Mac \text{-} {1}^{+ hi} \times 100}{Mac \text{-} {1}^{+ lo} + Mac \text{-} {1}^{+ hi}}$$

This measure was linear up to about 80% activation.
Isolation of Lipopolysaccharide (LPS) from Pyrularia-Associated Bacteria

LPS was extracted either from the Pyrularia fruit-fraction extract (LPS-FF) or from the isolated bacteria (ATCC deposit PTA-10285, deposited on Aug. 18, 2009) identified as Pantoea ananatis (LPS-P) by the method of Yi and Hackett (Analyst 125: 651-656, 2000) using Tri-Reagent (Molecular Research Center, Cincinnati, Ohio). Crude fractions were re-purified by the method of Manthey and Vogel (J Endotoxin Res 1-84-91, 1994) as detailed in Manthey et al. (J Immunol 153:2653, 1994) to remove LPS-associated proteins.

In vitro, in vivo, and ex vivo (bone marrow assay) procedures documenting the presence of lipopolysaccharide (LPS)

1. Limulus amoebocyte lysate (LAL) assay
a. Semiquantitative coagulation assay: LPS was assayed using the E-Toxate procedure (Sigma Chemical). This is a semi-quantitative method which relies on the presence of LPS for gel formation as a positive end-point.
b. Chromogenic endpoint: LPS-associated endotoxin units were quantitated by this variation of the LAL assay that is based on the enzymatic release of a chromophore in the presence of LPS. Kit QCL-1000 materials were obtained from BioWhittaker (Walkersville, Md.). Endotoxin units (EU) were based on standard endotoxin preparation from E. coli (0111:B4) which was supplied with the kit.

2. Reversal of bone marrow stimulation by poylmyxin

Polymyxin B is an antibiotic, which has strong affinity for LPS. Reversal of bioactivity in the presence of 10 μg/ml of polymyxin B was used as an indication of LPS-dependent bioactivity.

3. Quantitation of LPS by assay for sub-structural 2-keto-3-deoxyoctonate (KDO)

The thiobarbituric acid assay for KDO determination was done by the procedure of Weissbach and Hurwitz (J. Biol. Chem. 234:705-709, 1959) as modified by Karkhanis et al. (Anal. Biochem. 85:595-601, 1978).

### Nitric Oxide (NO) Assay

Nitric oxide was estimated by quantitation of nitrite in cell medium by the Griess reagent (1% sulfanimlamide/0.1% N-(1-napththyl)ethylenediamine dihdrochloride/2.5% H₃PO₄) as described by Green et al. (Anal. Biochem. 126:131-138, 1982) and modified for 96-well format as described by Jun et al. (Cellular Immunol 176:41-49, 1997).

### TNFα Assay

Cell supernatants were assayed for TNFα bioactivity in a cytotoxicity assay (described above) using actinomycin D-treated L929 cells as described by Hogan and Vogel (J. Immunol. 141:4196, 1988).

### Example 2

### Generation of Pyrularia Extracts (PyEx)

Pyrularia pubera is a terrestrial parasitic plant, which grows on the roots of oak trees. The material of this investigation was harvested from Cane Creek in North Carolina. Fruit was harvested during the fall season when the fruit was mature. The plant material was shipped on ice in an insulated container and immediately stored at −20° C.

Crude extracts were prepared from Pyrularia pubera tissues in the following manner:

Extraction from Fruit

between 250-500 grams of fruit was surface-sterilized with 70% etoh and allowed to thaw at room temperature overnight. The fleshy material was separated from the nut and blended thoroughly until a thin purée was obtained. Distilled water (100-200 mL, adjusted to pH 8.5 with ammonium hydroxide) was added to achieve a workable consistency. The solids were pelleted by centrifugation (12,000 rpm for 30 minutes) at 4° C. by means of a Sorvall RC-5B centrifuge (DuPont Instruments). The supernatant containing the activity was decanted into a separate container and further fractionated by ammonium sulfate precipitation as described below.

Extraction from Root

Pyrularia roots were washed in water to remove any dirt and debris and then blotted dry. The roots were shaved with a vegetable peeler into small, thin strips until the inner root core is exposed. The pieces were then freeze-dried and ground into a powder using a mechanical grinder. Lyophilized root powder was treated at −20° C. with acetone (5 m/g powder) with agitation for one hour to remove lipid material, then centrifuged at 4° C. for 5 minutes to pellet solid material, which was treated again with acetone overnight at −20° C. After collecting the solid by centrifugation, the supernatant liquid was removed and the root powder solid was allowed to air-dry. Root powder was extracted with 0.05 M sodium phosphate, pH 8.5 (10 ml/g root powder) for 3-15 hours at room temperature with stirring. The resulting slurry was centrifuged for 40 minutes at 15,000 rpm. The supernatant was retained and the solids were re-extracted with buffer. The first and second extracts were combined. Activity was then fractionated with ammonium sulfate precipitation as described below.

### Ammonium Sulfate Precipitation

An amount of ammonium sulfate sufficient to provide a 15% wt/wt saturation was slowly added with stirring to the supernatant. The mixture was allowed to stir at room temperature for 30 minutes and then centrifuged at 12,000 rpm for 30 minutes. The supernatant was collected and ammonium sulfate added to bring the liquid to 25% saturation. The mixture was stirred and centrifuged as above. The pellet obtained after centrifugation was solubilized in water and dialyzed (12-14 kD MWCO) against distilled water (pH 5-6) at 4° C. The initial dialysis was performed for 18 hours, then twice for two hours each against fresh distilled water. The dialyzed samples were concentrated on a Büchi Rotavapor R110 (Brinkmann Instruments) and lyophilized on a SpeedVac Concentrator (Savant) to obtain a light brown solid. The active material was usually enriched in the 15-25% ammonium sulfate cut. Following ammonium sulfate precipitation, the activity was dialyzed (12-14 LD MN cutoff) and lyophilized. This material is referred to as PyEx-1.

### Size-Exclusion Chromatography

Sephadex G75-Active lyophilized PyEx (20 mg/ml) was dissolved in water, adjusted to pH 8.5 with dilute ammonium hydroxide and placed onto a Sephadex G75-120 (Sigma Chemical, St. Lois, Mo.) column which had been pre-equilibrated with ammonia-water, pH 8.5. Initial elution was with water (pH 10). Fractions (1.5-2.0 ml) were collected starting before the elution of the colored material. Absorbance was monitored at 280 nm. Fractions were collected based on the various peaks. The desired activity was retained in the high molecular weight material eluting in the first 10 to 15 fractions. This fraction was designated PyEx-SEC.1 and contained the immunostimulatory activity and also contained cytotoxic activity.

### Anion-Exchange Chromatography

The PyEx-SEC.1 material was applied to an Econ Pac anion exchange cartridge (Bio-Rad, Hercules, Calif.) and eluted with a step gradient of low salt (10 mM ammonium carbonate) and then high salt (1M NaCl). The immunostimulatory activity was found predominantly in the low salt fraction (designated PyEx-AX.1A) and the cytotoxic activity was found predominately in the high salt fraction (designated PyEx-AX.1B). The cytotoxic activity was found to be heat labile (60° C., 10 minutes)

### FPLC Anion-Exchange Chromatography

A Uno FPLC anion-exchange column (Bio-Rad) was used to obtain a further purified fraction with immunostimulatory activity. The column was conditioned with Buffer A (20 mM Tris-Cl, pH 8.2). Following injection of PyEx material, activity was eluted at 2.0 ml/minute with Buffer A for 2.5 minutes, then with a linear gradient to 50% Buffer A+50% Buffer B (20 mM Tris-Cl, 1M NaCl, pH 8.2) over 10 minutes, then a linear gradient to 100% Buffer B over 2.5 minutes followed by an isocratic wash at 100% Buffer B. Fractions were grouped according to protein content (absorbance at 280 nm). Bioassay using the bone marrow activation assay revealed some activity in all fractions, but was highest in the fourth of six peaks. This material was designated PyEx-2.

### Example 3

### Physical Analysis of the Active Factors in PyEx

The immunostimulatory activity found in PyEx-2 was found to be stable to boiling, stable to treatment with detergent (0.5% sodium dodecyl sulfate, SDS) or chaotropic agents (6M guanidinium isothicyanate) and was also stable over pH 3-10 at 25° C. In addition, the activity was resistant to the effects of trypsin and proteinase K. Thus, treatment with proteinase K (50 μg/ml) in 10 mM Tris (pH 8.0), 10 mM EDTA, 0.5% SDS at 56° C. for 30 minutes had little effect on mitogenic activity but was sufficient to completely inactivate a similarly treated sample of concanavalin A. However, when subject to more severe (proteinase K at 2 mg/ml) and optimal conditions (1 mM Ca⁺⁺) for three hours at 37° C., the mitogenic activity in PyEx-2 was diminished by about 50%. Collectively, these findings suggested that the primary activity was probably not protein in nature or if protein is associated with or modulatory to the activity, it is not readily susceptible to proteolytic inactivation. It was subsequently found that the activity could be partially extracted into hot MeOH or BuOH.

### Example 4

### In Vitro Stimulation of Mouse Granulocytes

This example demonstrates that Pyrularia extracts, both PyEx-1 and PyEx-2, can stimulate granulocytes in vitro. This provides an assay useful for determining the relative immunostimulation provided by an extract or component of an extract, as described herein.

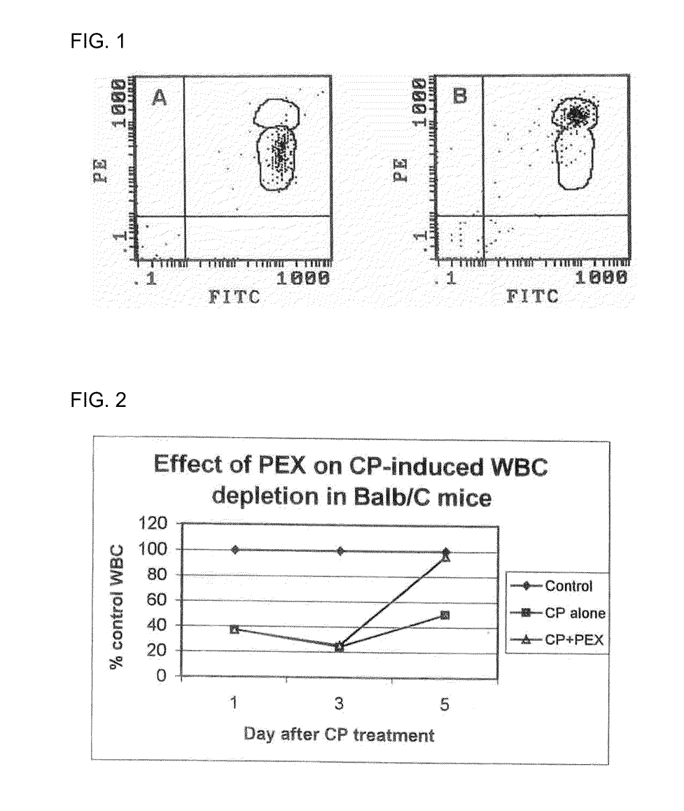

Mouse myelocytes were prepared as described above (Example 1), and responses of these cells to treatment with Pyrularia extract was examined using flow cytometry. The effect of PyEx-2 on stimulation of murine granulocytes in vitro is shown in FIG. 1. After 40 hours in the presence of PyEx-2 (2 μg/mL), the control unstimulated population of Gr-1⁺×Mac-1⁺ (Mac-1^{+lo}) population of myelocytes had migrated to a more intensely stained population along the y-axis (Mac-1^{+lo}). Prior to stimulation, 64% of myelocytes were Mac-1^{+lo}, with only 3% recognized in the “hi” region. Following PyEx-2 treatment, however, 43.8% of cells were in the activated state (see FIG. 1B). FIG. 1 shows results of the stimulation of murine bone marrow cells by PyEx-2.

### Example 5

### PyEx Prevents Chemotherapeutic-Drug Induced Death

This example demonstrates that Pyrularia extract functions in vivo to stimulate myelocytes, as measured by increased survival after administration of a chemotherapeutic drug, melphalan.

The effect of PyEx upon myelocyte stimulation was also observed in vivo as prevention of drug-induced myelosuppression. Balb/C mice were treated on day zero with an LD₅₀ dose of melphalan (100 mg/kg, i.p.). A separate group received melphalan (100 mg/kg, i.p.) and PyEx-1 (10 μg/mouse, subcutaneously). PyEx-1 was given immediately after or as late as 18 hours after melphalan administration, and every 48 hours thereafter.

TABLE 1
Prevention of drug-induced toxic death of Balb/C
mice by treatment with PyEx-1

| | | Drug related | Average time |
|---|---|---|---|
| Treatment group | Dose | deaths/# in group | of death |
| Melphalan | 100 mg/kg | 3/6 | day 7 |
| Melphalan + PyEx | 100 mg/kg | 0/6* | |
| | 10 μg/mouse | | |

*There were no drug-related deaths in the melphalan + PyEx-1 group at day 45, when the experiment was terminated.

As shown in Table 1, when mice were treated with an LD₅₀ dose of melphalan, lethal toxicity occurred on day eight in the melphalan alone group. However, in the group which received both melphalan and PyEx-1 (10 μg, every other day), no drug-related deaths occurred even at the time of termination of the experiment on day 45.

### Example 6

### In Vivo Neutropenia Model

This example demonstrates that PyEx stimulates white blood cells, particularly granulocytes such as neutrophils, after a sub-lethal dosage of the chemotherapeutic drug cyclophosphamide (CP).

Mice were injected on day 0 with a sub-lethal dose of cyclophosphamide (200 mg/kg). Eighteen to twenty-four hours later (day 1), control mice received an injection of saline and the PyEx group received a single dose of PyEx (20 μg/mouse). Mice were bled sequentially via the retro-orbital sinus or tail vein at various times thereafter. Blood was collected in heparinized capillary tubes.

For determination of total versus white cells, blood was diluted into Isoton II (Beckman-Coulter, Fullerton, Calif.). Total cells and white cells were counted using a Model ZM Coulter counter (Beckman-Coulter, Fullerton, Calif.). White cell counts were determined following treatment of whole blood with a RBC lytic reagent (Zap-Oglobin, Beckman-Coulter, Fullerton, Calif.). Blood granulocytes and myelocytes were quantitated by means of an EPICS-XL™ flow cytometry after lysis of red cells.

As shown in FIG. 2, total WBC counts reached the nadir on the third day following CP treatment. By day five, the total WBC count was restored by simultaneous treatment with PyEx, whereas the CP alone level was only partially restored (to 45% of the difference between the nadir value and the control value). This effect is seen after a single dose of PyEx-1 delivered 24 hours after CP treatment.

To more clearly determine what populations of cells were stimulated by treatment with PyEx, in a similar experiment blood and bone marrow cells were collected and analyzed for total blood granulocytes and myelocytes. Balb/C mice were treated with cyclophosphamide (200 mg/kg) on Day 0. PyEx-1 (20 μg/mouse) was given to mice 24 hours after administration of CP. Blood from PyEx-1 treated experimental and saline treated control mice was collected with heparinized capillary tubes five days after CP administration. Following lysis of red cells, the remaining cells were analyzed by means of an EPICS-XL™ flow cytometer (Beckman-Coulter, Fullerton, Calif.). Mature granulocytes were identified based on forward versus side scan profile set for control (untreated) mouse blood.

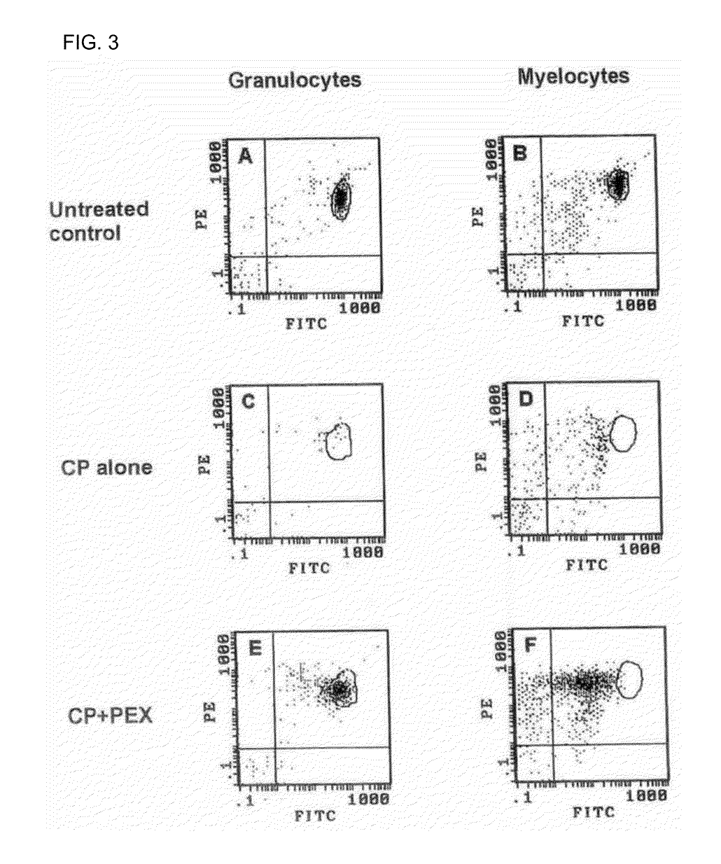

On day five after CP treatment, blood and marrow cells were collected and analyzed for total blood granulocytes and myelocytes. As shown in FIG. 3 and quantitated in Table 2, blood granulocytes were severely depleted (<2% of control) by CP treatment by day five, but were restored to 130% of the control value by treatment with a single dose of PyEx, administered one day after CP.

TABLE 2
Restoration of blood granulocytes following
sub-lethal chemotherapy of Balb/C mice

| | | Percentage of |
|---|---|---|
| | | Granulocytes |
| Treatment Group | Dose | in Control |
| Cyclophosphamide | 200 mg/kg | 1 |
| Cyclophosphamide + | 200 mg/kg | 130 |
| PyEx-1 | 20 μg/mouse | |

### Example 7

### Activation of Human Bone Marrow

This example demonstrates that the Pyrularia fruit extract can effectively stimulate human bone marrow cells. In addition to illustrating the usefulness of Pyrularia fruit extract as a clinical immunostimulant for use with human subjects, this example also provides an additional system in which immunostimulatory activity of a Pyrularia extract, or component thereof, can be tested.

Human bone marrow was provided by the Bone Marrow Transplantation Unit, University of Utah. Experiments were initiated upon obtaining fresh marrow from normal donors. Whole marrow was treated as described above for murine marrow samples. Following incubation for 24 to 36 hours with PyEx or other agents as described, cells were stained with anti-Mac-1-FITC×anti-CD14-PE. Cells were analyzed using a Coulter EPICS-XL™ flow cytometer (Beckman-Coulter, Fullerton, Calif.). Activated populations were identified by positive staining with Mac-1×CD14 (designated Mac-1⁺, CD14⁺).

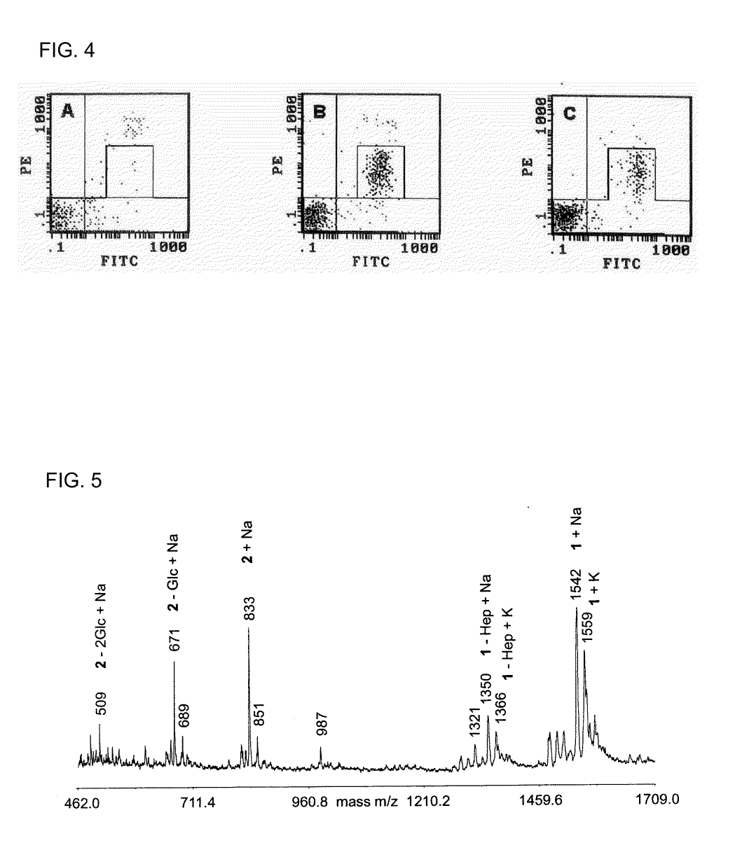

PyEx-2 was capable of stimulating human myelocytes, as shown in FIG. 4. Fresh human marrow from a normal donor was incubated without drug, or in the presence of PyEx-2 or rhGM-CSF, and the activating effect was quantitated by staining with anti-MAC-1-FITC×anti-CD14-PE. As shown, both the PyEx-2 and rhGM-CSF treatments produced an activated sub-population of myelocytes, with the PyEx-2 sub-population containing 14% of total cells and the GM-CSF-treated cells containing a new population with 9% of the total cells.

### Example 8

### LPS-Related Assays

This example demonstrates the involvement of LPS in the PyEx preparation. Polymyxin B is an antibiotic which is effective against gram negative bacteria by being able to bind tightly to the lipid A portion of bacterial LPS and prevent binding to cellular receptors. Reversal of immunostimulatory activity on addition of polymyxin B to an assay is considered to be indicative of the involvement of LPS. As shown in Table 3, 62% of murine bone marrow cells treated with PyEx were ‘activated.’ However, in the presence of polymyxin B, only 29% of the cells were activated, suggesting that a substantial portion of this activity was a result of PyEx-associated LPS. A similar result was seen on treatment with LPS extracted from plant-free bacteria, LPS-P.

TABLE 3
Reversal of PyEx-dependent murine
bone marrow by polymyxin B

Percent activation

| Treatment | Conc. | No polymyxin | 10 μg/ml polymyxin |
|---|---|---|---|
| Control | — | 15.0 | 13.6 |
| PyEx | 0.5 μg/ml | 62.3 | 28.7 |
| LPS-P | 0.45 μg/ml | 62.2 | 23.5 |

Further direct and indirect assays were used to verify the presence of LPS and quantitate the amount of LPS present in various preparations. Two functional assays were compared with two assays that have been used to quantify the relative amounts of LPS in various samples. The functional assays are based on the LPS-dependent activation of a Limulus amoeboctye lysate (LAL), but with two different end points. The first assay (E-Toxate, Sigma Chemical) was based on a gelation end point and is semi-quantitative. Commercial LPS from S. typhosa gave a positive test, but PyEx tested negative. The second assay was a LAL-based assay but with a chromogenic endpoint and is quantitative. The results are shown in Table 4 and are expressed in endotoxin units (EU). PyEx possessed 940 EU/μg of material, which was approximately one third the 2600 EU/μg associated with a commercial LPS and was three-times that of a purified LPS from Pyrularia-associated bacteria (LPS-P, 312 EU/μg). These relative amounts were in good agreement with those of an assay to determine sub-structural components of LPS. Thus, a thiobarbituric acid assay was used to detect KDO found in the core region of LPS. These results, also shown in Table 4, were in agreement with the LAL assay. PyEx contained 4.7 μg KDO/mg of extract which was about one third of the 13.8 μg/KDO/mg of a commercial preparation, but it was only a little more than twice the amount from LPS-P.

TABLE 4
Quantitation of LPS in PyEx and LPS-P by a
functional LAL assay and by direct measurement
of 2-keto-3-deoxyoctonate (KDO)

| Treatment | LAL (EU/μg) | KDO (μg/mg) |
|---|---|---|
| PyEx | 940 | 4.7 |
| LPS-P | 312 | 2.1 |
| LPS-S. typhosa | 2600 | 13.8 |

### Example 9

### Generation of TNFα and NO in Bone Marrow Cells and Macrophages

One concern with the use of an LPS-containing material is the uncontrolled-toxicity associated with high doses of LPS, which is attributed to uncontrolled production of TNFα or nitric oxide (NO). Although no overt toxicity has been observed in animals injected with PyEx (40 μg/mouse; every other day for over 6 weeks), the effect of PyEx on these two immune mediators is shown in Table 5 for peritoneal exudate cells (PEC), a macrophage population which has been ‘primed’ by injection with thioglycollate, and in Table 6 for bone marrow cells. TNFα and NO were assayed as described above. In the case of NO, the stable metabolite NO₂ was assayed and is used as an indication of the amount of NO formed. High NO levels are normally found only in the presence of INFγ. However, bone marrow cells treated with PyEx were found to generate low levels of NO without added INFγ. In contrast, little TNFα was detected in bone marrow, but higher amounts were found in PEC cultures treated with PyEx or LPS-P.

TNFα formation was attenuated by treatment with INFγ and completely prevented with 1 mM pentoxifylline, a dose which also diminished the amount of NO formed by PEC. Pentoxifylline may also show a similar effect to reverse the formation of NO in bone marrow cells; however, this has not been demonstrated. A higher dose of pentoxifylline did not prevent bone marrow activation (see Example 10).

TABLE 5
Levels of TNFα and NO generated on treatment of
PEC with PyEx, LPS-P, and with PyEx in the presence
of IFNγ and pentoxifylline

| Treatment | Dose | NO₂ (μM) | TNFα (ng/ml) |
|---|---|---|---|
| PyEx | 0.1 μg/ml | 0.06 | 1.43 |
| LPS-P | 0.1 μg/ml | 0.00 | 1.8 |
| PyEx | (0.1 μg/ml) + INFγ (5 U/ml) | 11.02 | 0.51 |
| PyEx | (0.1 μg/ml) + INFγ (5 U/ml) + | 6.40 | 0.00 |
| | pentoxifylline (1 mM) | | |

TABLE 6
Levels of TNFα and NO generated on treatment of bone marrow
cells with PyEx and with PyEx in the presence of IFNγ

| Treatment | Dose | NO₂ (μM) | TNFα (ng/ml) |
|---|---|---|---|
| PyEx | 0.5 μg/ml | 0.74 | 0.01 |
| INFγ | 5 U/ml | 0.00 | 0.00 |
| PyEx | (0.5 μg/ml) + INFγ (5 U/ml) | 17.19 | 0.07 |

### Example 10

### Effect of Pentoxifylline on PyEx-Induced Bone Marrow Activation

This example shows that, while certain agents such as pentoxifylline can attenuate the formation of certain toxic immune modulators (Example 9), a much higher dose of pentoxifylline (3 mM) did not abolish the bone marrow activating effect of PyEx (Table 7). In comparison with a PyEx control, the percent of cells activated in the assay was attenuated from 63 to 54%. The same degree of attenuation was also seen between the control and pentoxifylline groups, suggesting that the effect was not directly on bone marrow activation, but rather on an underlying mechanism which gives rise to a natural level of activated cells. In addition, this dose of pentoxyfylline showed some toxicity as indicated by the decreased viability of the bone marrow cultures. Together these results suggest that agents such as pentoxifylline may be used to attenuate the elaboration of toxic immune mediators from, for example, macrophage populations, while allowing the activation of the bone marrow granulocyte population.

TABLE 7
Continued effect of PyEx on bone marrow cell activation
in the presence of pentoxifylline

| Treatment | Dose | Viability (%) | Activation (%) |
|---|---|---|---|
| Control | — | 76.9 | 17.5 |
| PyEx | 0.5 μg/ml | 79.5 | 62.9 |
| pentoxifylline | 3 mM | 61.5 | 7.54 |
| PyEx + | 0.5 μg/ml + 3 mM | 59.5 | 54.03 |
| pentoxifylline | | | |

### Example 11

### Effect of PyEx on the Activity of Other LPS Preparations

Fresh bone marrow cells were cultured with PyEx or certain commercial preparations of LPS including those from Salmonella minnesota (Sigma Chemical L-6261), Salmonella typhosa (Sigma L-7136), Escherichia coli (Sigma L-3254) or Serratia marcescens (Sigma L-6136). These commercial preparations are either phenol or TCA extracts. PyEx was present at 0.5 μg/ml. Commercial preparations were each tested separately at 0.1 μg/ml and then in combination with PyEx at 0.5 μg/ml.

The results are shown in Table 8. In three cases, combining the various commercial preparations with PyEx (0.5 μg/ml) provided little additional activation beyond that obtained with PyEx alone (72%). In the case of S. typhosa which had the highest activity (88%) as a single agent, the degree of activation was highest (80%) among the combinations, but was considerably lower than what may be expected for any additive effect and indeed this appeared to be an antagonistic combination. These results indicate a novel dominant feature of PyEx when tested in combination with other LPS preparations.

TABLE 8
Effect of PyEx on the bone marrow (BM) cell
activation induced by other LPS preparations

| | | Percent BM cell activation | |
|---|---|---|---|
| LPS source | Conc. | Without PyEx | PyEx (0.5 μg/ml) |
| Control | — | 15.1 | 72.2 |
| S. minnesota | 0.1 μg/ml | 56.8 | 76.8 |
| S. typhosa | 0.1 μg/ml | 88.4 | 80.4 |
| E. coli | 0.1 μg/ml | 23.2 | 75.4 |
| S. marcescens | 0.1 μg/ml | 71.2 | 78.6 |

Over 15×10³ cells were analyzed for each sample. Bone marrow cell viability among all samples including the untreated control was 70-76% as determined by propidium iodide exclusion.

### Example 12

### Testing of LPS Samples Obtained from Pyrularia-Associated Bacteria

This example demonstrates that LPS obtained by solvent extraction of bacteria associated with Pyrularia fruit can activate bone marrow cells. The solvent extraction procedure is described below in Example 13. Two different LPS samples were prepared and designated sample #1 and sample #2.

### Procedure

Balb/C mouse bone marrow cells derived from a 7 week old animal was activated with LPS at 1.2×10⁶ cells/ml in RPMI-1640 containing 10% fetal calf serum, L-glutamine, and Pen/Strep for 18 hours at 37° C. and 5% CO₂. Cells were harvested, blocked with CD16/32 antibody and stained with Rat IgG2b FITC, Rat IgG2b PE, CD11b (M1/70), and Gr-1 (RB6-8C5).

### Results

There was virtually no change in the percentage (or the mean fluorescence intensity) of cells expressing Gr-1 alone or CD11b alone in cells stimulated with either LPS sample compared to media control. In addition, there was virtually no change in the mean fluorescence intensity of the Gr-1 positive cells contained in the double-positive population (Gr-1⁺/CD11b⁺). Differences were observed, however, in the percentage of cells in the Gr-1/CD11b double positive population as well as the mean fluorescence intensity of CD11b staining within this double-positive population.

Compared to the media control, stimulation with either LPS sample caused a decrease in the total percentage of Gr-1⁺/CD11b⁺ cells. The percentage of Gr-1⁺/CD11b⁺ cells was reduced from 32.8% in the media control to 13-24% in LPS sample #1 (Table 9) and 18.9-26.1% in LPS sample #2 (Table 10).

Relative to the media control, both LPS samples resulted in an increase in the mean fluorescence intensity of CD11b in the Gr-1⁺/CD11b⁺ population, which is indicative of an upregulation of CD11b on a per cell basis. In LPS sample #1, this increase was approximately 3-fold or greater (depending on the dose) and in LPS sample #2 this increase was approximately 2-fold or greater (depending on the dose). Overall, these effects did not show a clear dose dependency but were lower in the highest concentration of LPS used (200 μg/ml).

TABLE 9
Stimulation of BM cells with LPS Sample #1

| LPS #1 | | | MFI in CD11b⁺/Gr-1⁺ |
|---|---|---|---|
| Sample | | Gr-1⁺/CD11b⁺ (%) | population |
| Control | | 32.8 | 501 |
| 200 | μg/ml | 13.2 | 1408 |
| 50 | μg/ml | 18.6 | 1889 |
| 12.5 | μg/ml | 17.8 | 1856 |
| 3.1 | μg/ml | 18.2 | 1774 |
| 0.78 | μg/ml | 24.43 | 1827 |
| N—F¹ 50 | μg/ml | 14.77 | 2168 |

¹As a control, one sample of non-filtered (N—F) LPS#1 was added to BM cells

TABLE 10
Stimulation of BM cells with LPS Sample #2

| LPS #2 | | | MFI in CD11b⁺/Gr-1⁺ |
|---|---|---|---|
| Sample | | Gr-1⁺/CD11b⁺ (%) | population |
| Control | | 32.8 | 501 |
| 200 | μg/ml | 26.1 | 993 |
| 50 | μg/ml | 18.9 | 1187 |
| 12.5 | μg/ml | 20.1 | 1214 |
| 3.1 | μg/ml | 19.9 | 1316 |
| 0.78 | μg/ml | 21.4 | 1275 |

These results demonstrate that neither LPS sample #1 nor LPS sample #2 changed the number of cell expressing either Gr-1 or CD11b. However, in cells expressing both Gr-1 and CD11b, the expression of CD11b was increased 2- to 3-fold. Moreover, the LPS was very potent, as a dose-dependent response was not observed, even at the lowest concentration of LPS tested (0.78 μg/ml). Both preparations of the LPS were active for stimulation of bone marrow cells.

### Example 13

### Production and Extraction of LPS from Pyrularia-Associated Bacteria

This example describes the growth of Pyrularia-associated bacteria and subsequent isolation of LPS from the bacteria for analysis of LPS activity (Example 14) and determination of the LPS core structure (Example 15).

### Bacteria Production

Isolated Pyrularia-associated bacteria were used to inoculate 50 mL of LB broth. The inoculated broth was incubated at 27° C. overnight with shaking. Frozen stocks of bacteria were thawed and seeded into four shaker flasks containing LB and placed in a shaker box at 30° C. A 10 L fermenter was prepared and ready for media prior to thawing bacteria. Modified media was prepared and placed in the fermenter prior to inoculation to ensure sterility. A total of 9 L of FB2 media was prepared according to Liu et al. (Proc. Natl. Sci. Counc. Repub. China B. 24(4):156-160, 2000). Table 11 provides the components of FB2 media per 1 L.

TABLE 11
Components of FB2 Media

| Part | Component | Volume or Weight | |
|---|---|---|---|
| Trace Elements | dH₂O | 60 | mL |
| | FeCl₂•6H₂O | 2.7 | g |
| | ZnCl₂ | 0.13 | g |
| | CoCl₂•6H₂O | 0.2 | g |
| | Na₂MoO₄•2H₂O | 0.2 | g |
| | CaCl₂•2H₂O | 0.1 | g |
| | CuCl₂•2H₂O | 0.13 | g |
| | H₃BO₃ | 0.05 | g |
| | Conc. HCl | 10 | mL |
| Part 1 | dH₂O | 500 | mL |
| | Yeast Extract | 5 | g |
| | N-Z Anime HD | 5 | g |
| Part 2 | dH₂O | 300 | mL |
| | Sorbitol | 40 | g |
| | Trace element | 3 | mL |
| | CaCl₂•2H₂O | 0.03 | g |
| | (Na₄)₂SO₄ | 1.2 | g |
| | Na₄Cl | 0.2 | g |
| | MgSO₄ | 1.17 | g |
| Part 3 | dH₂O | 200 | mL |
| | KH₂PO₄ | 4 | g |
| | K₂HPO₄ | 4 | g |
| | Na₂HPO₄ | 2.8 | g |

Silicon antifoam 1520 (Dow Corning Corporation, Midland, Mich.) was prepared according to the manufacturer's instructions. Parts 1-3 of the FB2 media and the prepared antifoam were autoclaved at 121° C. for 60 minutes. FB2 media was pumped into the 10 L fermenter sequentially beginning with Part 1 and ending with Part 3. The media was adjusted to the appropriate temperature (37° C.) and pH (6.5) and left overnight prior to inoculation with 2 L of cultured bacteria (OD₆₀₀=1.0).

Bacteria was harvested at OD₆₀₀˜10.0 and concentrated to approximately 2-3 L with a 0.2 μM Cellflo™ filter (Spectrum Labs, Rancho Dominguez, Calif.). Concentrated material was spun down at 5,000 rpm for one hour at 4° C. Wet cell paste was then weighed and frozen at −20° C. prior to the extraction process.

### LPS Extraction

Wet cell paste was resuspended with a single phase Bligh-Dyer (SPBD) (Bligh and Dyer, Can. J. Biochem. Physiol. 37(8):911-917, 1959) mixture (approximately 5 mL SPBD per gram of wet cell paste) and placed in a shaker box for 90 minutes at room temperature. After shaking, the mixture was centrifuged at 3,000 rpm for 10 minutes and the supernatant discarded. The pellet was resuspended with fresh SPDB mixture and stirred for one hour. After stirring, the mixture was centrifuged at 3,000 rpm for 10 minutes and the supernatant was discarded. The wet cell paste was then subjected to phenol-water extraction as follows.

The wet cell paste was stirred with deionized water and heated to 65-68° C. using a water bath. Once heated, an equal volume of pre-warmed 90% phenol was added. The 1:1 phenol water mixture was stirred vigorously for 20 minutes at 70-80° C. The phenol/water/cell paste mixture was then cooled in an ice bath to 2° C. and dialyzed against deionized water at 4° C. for 24 hours. After 24 hours, the dialyzed emulsion was centrifuged for 15 minutes at 6,000×g at 4° C. The upper water phase was siphoned off and dialyzed for 72-96 hours against deionized water at 4° C. The dialyzed material was lyophilized, weighted and assayed for purity and quality. In total, 400 mg of LPS was extracted from 57 grams of wet cell paste.

### Analysis

An LAL assay was performed using standard LPS from Escherichia coli. The results showed that the LPS extracted from Pyrularia-associated bacteria was pyrogenic. In addition, the extracted LPS preparation contained no detectable protein and little nucleic acid as determined by UV absorption at 256 nm. Evaluation of the purified LPS by SDS-PAGE and silver stain demonstrated that the preparation contained no detectable protein and exhibited a similar pattern to E. coli LPS. FTIR analysis also demonstrated that the purified LPS from Pyrularia-associated bacteria was similar in structure to E. coli LPS.

### Example 14

### Evaluation of LPS Activity at Increasing Levels of LPS Purity

This example demonstrates that increasing the purity of LPS isolated from Pyrularia-associated bacteria does not decrease the immunostimulatory activity of the LPS. These results suggest that it is the LPS, and not other contaminating factors, that possesses the immunostimulatory activity.

### Procedure

Bone marrow cells (1.5×10⁶ cells/ml) isolated from 7 week old Balb/c mice were stimulated with the indicated concentration of LPS in RPMI 1640 medium containing 10% FBS, 2 mM L-glutamine, and 1% pen/strep for 20 hours at 37° C. and 5% CO₂. Cells were harvested, Fc receptor was blocked with CD16/32 antibody and the cells were stained with following antibodies: rat IgG2b F, rat IgG2b PE, CD11b (M1/70) PE, and Gr-1 (RB6-8C5) F.

### Results

There was no significant change in the mean fluorescence intensity (MFI) of Gr-1 staining in the Gr-1/CD11b double positive population in the purified LPS stimulated samples compared to media control. However, the MFI of CD11b staining increased in the double positive population stimulated with the purified LPS at each dilution, relative to the media control (see Table 13 below).

Compared with the media control, the crude LPS stimulation (especially from 1:20 to 1:320 dilution) resulted in a decreased percentage of the Gr-1/CD11b double positive population (from 20.4% to 10.8% at 1:20 dilution) and an increased MFI of CD11b staining at all LPS dilutions (see Table 14 below)

TABLE 13
Purified LPS

| LPS Dilution | CD11b MFI | Gr-1 MFI | % Double-positive |
|---|---|---|---|
| Media | 286.4 | 2672.1 | 20.4 |
| 1:20 | 816.1 | 2317.1 | 13.8 |
| 1:80 | 968.9 | 2732.1 | 11.2 |
| 1:320 | 826.1 | 2450.7 | 15.6 |
| 1:1280 | 864.3 | 2752.5 | 13.3 |
| 1:5120 | 806.1 | 2807.0 | 19.3 |
| 1:20480 | 566.2 | 2413.7 | 15.3 |

TABLE 14
Crude LPS

| LPS Dilution | CD11b MFI | Gr-1 MFI | % Double-positive |
|---|---|---|---|
| Media | 286.4 | 2672.1 | 20.4 |
| 1:20 | 851.7 | 2797.8 | 10.8 |
| 1:80 | 911.4 | 2846.7 | 9.4 |
| 1:320 | 900.7 | 2800.0 | 8.4 |
| 1:1280 | 899.2 | 2926.1 | 12.8 |
| 1:5120 | 772.3 | 3212.8 | 14.7 |
| 1:20480 | 691.5 | 2820.9 | 13.6 |

These results demonstrate that purification of the crude LPS does not inhibit immunostimulatory activity, indicating that the LPS is the active component.

### Example 15

### Analysis of the LPS Core Structure

This example describes the structure of the core of LPS isolated from Pyrularia-associated bacteria.

### Methods

### Nuclear Magnetic Resonance (NMR) Spectroscopy

NMR spectra were recorded at 25° C. in D₂O on Varian UNITY INOVA 600 instrument, using acetone as a reference for proton (2.225 ppm) and carbon (31.5 ppm) spectra. Varian standard programs COSY, NOESY (mixing time of 200 ms), TOCSY (spinlock time of 120 ms), HSQC, and gHMBC (long-range transfer delay of 100 ms) were used.

### Matrix-Assisted Laser Desorption Ionization (MALDI)

Mass spectra were obtained using Perseptive Biosystems Voyager DE STR spectrometer with 2,4-dihydroxybenzoic acid (DHB) matrix. Electrospray mass spectra were obtained using a Micromass Quattro spectrometer in 50% MeCN with 0.2% HCOOH at flow rate 15 mkl/min with direct injection.

### Monosaccharide Analysis

LPS or core (0.5 mg) was hydrolyzed (0.2 mL of 3M TFA, 120° C., 2 hours), followed by evaporation to dryness under a stream of air. The residue was dissolved in water (0.5 mL), reduced with NaBH₄ (approximately 5 mg, 1 hour), neutralized with AcOH (0.3 mL), dried, and MeOH (1 mL) was added. The mixture was dried twice with the addition of MeOH. The residue was acetylated with Ac₂O (0.5 mL, 100° C., 30 minutes), dried and analyzed by GLC on a HP1 capillary column (30 m×0.25 mm) with a flame ionization detector (Agilent 6850 chromatograph) in a temperature gradient from 170 to 260° C. at 4° C./minutes and by gas chromatography-mass spectrometry (GC-MS) on Varian Saturn 2000 ion-trap instrument on the same column.

### Gel Chromatography

Gel chromatography was carried out on Sephadex G-50 (2.5×80 cm) or Sephadex G-15 (1.6×80 cm) columns using the pyridinium acetate buffer, pH 4.5 (4 mL pyridine and 10 mL AcOH in 1 L water) as eluent, and monitored by a refractive index detector.

### Anion-Exchange Chromatography

Anion-exchange chromatography was performed on a Hitrap Q column (Pharmacia) in water for 10 minutes, then in a linear gradient of 0 to 1 M NaCl over 60 minutes with UV detection at 220 nm. Fractions were desalted by gel chromatography on Sephadex G-15 column.

### Preparation of the Core

LPS (25 mg) was hydrolyzed with 2 mL 2% AcOH for 3 hours at 100° C. The precipitate of the lipid was removed by centrifugation at 12000 rpm, and soluble products were separated by gel chromatography on a Sephadex G50 column. Polymeric and oligosaccharide fractions were obtained. Oligosaccharide (core) fraction was further separated on an anion-exchange column and desalted on Sephadex G-15.

### Deamination of the Core

Core (4 mg) was treated with 10% AcOH-5% NaNO₂ for 2 hours at 25° C. The oligosaccharide fraction was desalted by gel chromatography on Sephadex G15 column.

### O-Deacylation of the LPS

LPS (10 mg) was dissolved in anhydrous hydrazine, kept 1 hour at 50° C., cooled, and poured in cold acetone (50 mL). The precipitate was collected by centrifugation, washed with acetone, dissolved in water and lyophilized.

### Methylation Analysis

Core (1 mg) was dissolved in anhydrous DMSO, dry powdered NaOH (approximately 50 mg) was added, and the mixture was stirred for 1 hour. MeI (0.3 mL) was added, stirred for 30 minutes, and excess MeI was removed by air blow. The remainder diluted with water (5 mL) and extracted with CHCI₃ (5 mL). The organic layer was washed 3 times with water and dried. Methylated product was converted into alditol acetates as described for monosaccharide analysis using NaBD₄ and analyzed by GC-MS.

### Results

For the preparation of the polysaccharide and core oligosaccharide, LPS was hydrolyzed by 2% AcOH, soluble products were separated by gel chromatography, and the oligosaccharide fraction was further separated by anion-exchange chromatography to give pure core oligosaccharide. A small amount of polymeric material, eluted at the solvent front in gel chromatography, had a characteristic ¹H NMR spectrum of starch, often found in LPS preparations. No LPS-related polysaccharide was obtained, as expected from PAGE results showing no PS bands.

Monosaccharide analysis of the whole LPS and isolated core (GC-MS of alditol acetates) showed the presence of glucose, glucosamine, D-glycero-D-manno-heptose (referred to as DD-heptose or DD-Hep) and L-glycero-D-manno-heptose (referred to as LD-heptose or LD-Hep).

The LPS core was analyzed by NMR spectroscopy. A set of 2D spectra (COSY, TOCSY, NOESY, ¹H-¹³C HSQC, HMBC and HMQC-TOCSY) was recorded and interpreted using the Pronto program. Spectra were complex because of the presence of several structural variants, differing by the attachment of glucose residues at the non-reducing end (incomplete presence of Glc P, L and K, and Hep T), and the presence of several variants of KDO degradation products (“normal KDO”, 4,7- and 4,8-anhydro-derivatives) at the reducing end. Monosaccharides were identified using characteristic patterns of TOCSY and COSY cross peaks, as well as chemical shift data. Identity of galacturonic acid Y was determined using HMBC correlation between its H-5 and carboxyl group. High field position of 11-2 signal of GlcN X pointed to the absence of acyl substituent at its amino group. DD- and LD-heptoses were distinguished on the basis of chemical shifts of their C-6 (DO heptose has C-6 at ˜73 ppm, whereas LD heptose has this signal at ˜70 ppm); and H-5 signals (in DD-Hep they are at ˜3.8 ppm, and in LD-Hep at ˜3.6 ppm). Using these markers it was found that Hep S and T are DD, and all other Hep residues are LD.

The connection between monosaccharides was determined from NOE and HMBC data. The following NOE correlations were identified: E1C5,7; F1E3; G1F7; H1E4,6; G1F7; Y1F3; X1Y4,5; Z1X4; K1Z6; P1K1,2: L1K6; S1Y1,2; Y1T5; T1S1,2. Corresponding inter-residual HMBC were observed.

For independent linkage analysis, the core was methylated by the Ciucanu-Kerek procedure (Ciucanu and Kerck, Carbohydr. Res. 131:209-217, 1984) and hydrolyzed. Monosaccharides were reduced with NaBD₄, acetylated and analyzed by GC-MS. Derivatives of all neutral monosaccharides, expected from the presented structure, were detected: terminal, 2-, 6-, and 2,6-substituted glucopyranose, terminal DD- and LD-heptopyranose, 2-substituted DD-heptopyranose, 3,4- and 3,7-disubstituted heptopyranose.

The structure was confirmed by electrospray ionization-mass spectrometry (ESI-MS), which gave a mass of 2328 Da for the maximal structure and three peaks of species lacking glucose residues (162 units), corresponding to the absence of Glc L, P, and K. Structures without Hep were not identified in this mass spectrum because of low abundance.

For additional proof of the structure, core oligosaccharide was deaminated by NaNO₂—AcOH and the products analyzed by MALDI and ESI-MS. Expected cleavage of GlcN X (which was converted into anhydromannose) led to formation of oligosaccharides with maximal structures 1 and 2; both had smaller variants due to lack of Glc and Hep. Full oligosaccharide 1 with a mass of 1518.5 Da and a minor variant lacking Hep T with a mass of 1326.5 Da were visible in ESI and MALDI spectra. Oligosaccharide 2 had no charged groups and was visible only in MALDI spectrum (FIG. 5) as Na- and K-adducts, giving peaks (Na-adducts) at m/z 833, 671 (loss of one glucose), and 509 (loss of two glucose) in agreement with the proposed structure.

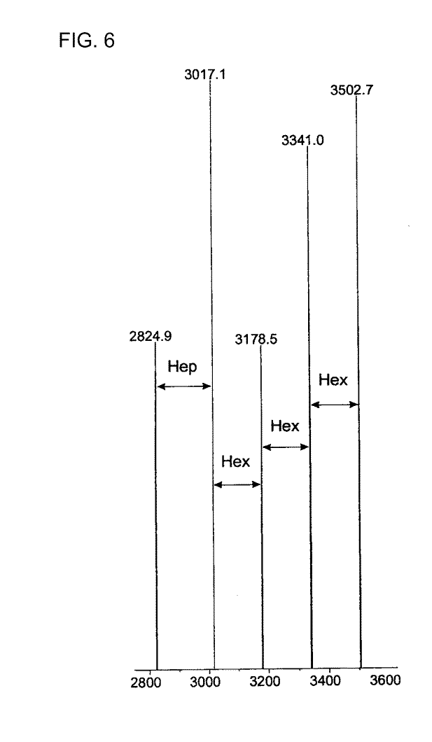

LPS was O-deacylated by anhydrous hydrazine treatment and analyzed by ESI-MS (FIG. 6). Maximal structure with a composition Hex₅Hep₅HexN₃HexA₁Kdo₂C14OH₂P₂ with a mass of 3502.7 Da (expected 3502.6) was present, as well as three products with sequential loss of glucose (162 Da) with masses of 3341.0, 3178.5, and 3017.1 Da. The variant with missing heptose was the only one with simultaneously missing three glucose residues (2824.9 Da). Thus, the LPS has a standard structure with two KDO residues and does not contain other acid-labile components, which could be lost during core preparation. The analyzed core structure has close resemblance to the Serratia marcescens core (Vinogradov et al., Chemistry 12(25):6692-700, 2006), differing by additions of glucose residues at the non-reducing end, and by replacement of LD-Hep T in Serratia with DD-Hep in the analyzed structure. Ko, replacing side-chain KDO in Serratia, was not detected in the analyzed product.

TABLE 12
NMR data for largest structure

| Unit | H/C 1 | H/C 2 | H/C 3 | H/C 4 | H/C 6 | H/C 6a | H/C 7/6b | H/C 7b/8 |
|---|---|---|---|---|---|---|---|---|
| F | 5.36 | 4.14 | 4.00 | 3.99 | 3.62 | 4.23 | | |
| | 102.5 | 71.5 | 81.9 | 66.7 | 73.0 | 69.3 | | |
| G | 4.94-4.97 | 3.99 | 3.87 | 3.88 | 3.64 | 4.06 | | |
| | 101.9 | 71.5 | 72.4 | 67.7 | 72.9 | 70.5 | | |
| H, β-Glc | 4.62-4.56 | 3.31 | 3.52 | 3.39 | 3.40 | 3.75 | 3.88 | |
| | 104.3 | 77.6 | 77.1 | 71.2 | 77.7 | 62.9 | | |
| K, β-Glc | 4.64 | 3.46 | 3.58 | 3.45 | 3.66 | 3.97 | 3.80 | |
| | 104.3 | 79.1 | 76.3 | 70.9 | 75.9 | 67.3 | | |
| P, α-Glc | 5.37 | 3.61 | 3.75 | 3.47 | 4.03 | | | |
| | 5.37 | 3.61 | 3.75 | 3.47 | 4.03 | | | |
| L, α-Glc | 4.97 | 3.57 | 3.73 | 3.44 | 3.73 | | | |
| | 99.4 | 73.2 | 73.5 | 71.0 | 74.8 | | | |
| Z, α-Glc | 5.50 | 3.61 | 3.74 | 3.54 | 3.86 | 3.89 | 4.16 | |
| | 100.9 | 73.2 | 74.4 | 70.9 | 73.0 | 69.8 | | |
| X, GlcN | 5.20 | 3.36 | 4.22 | 3.82 | 4.37 | 3.81 | 3.87 | |
| | 97.4 | 5.7 | 71.9 | 76.5 | 72.5 | 61.5 | | |
| Y, GalA | 5.49 | 4.10 | 4.23 | 4.46 | 4.51 | | | |
| | 100.0 | 72.9 | 68.4 | 80.5 | 73.8 | 176.7 | | |
| S, DDHep | 5.37 | 3.99 | 4.00 | 3.75 | 3.97 | 4.07 | | |
| | 96.8 | 80.7 | 71.5 | 68.8 | 75.3 | 72.7 | | |
| T, DDHep | 5.10 | 4.07 | 3.78 | 3.75 | 3.85 | 4.07 | | |
| | 104.1 | 71.6 | 72.4 | 68.8 | 75.8 | 72.7 | | |

* Italics indicates ¹³C chemical shifts

### Example 16

### Identification of Pyrularia-Associated Bacteria

This example describes the identification of the bacteria associated with Pyrularia fruit extracts. Bacteria were grown on LB plates by plating fruit extract and isolating single colonies. Single colonies were further expanded in 1 ml cultures of LB. Bacterial genomic DNA was isolated using the gram negative bacteria protocol of the Qiagen Blood and Tissue DNA Isolation Kit (Valencia, Calif.). Twenty μl of packed bacterial cell pellet was used for isolation. The following PCR primers were used for amplification of genomic DNA: TGGAGAGTTTGATCCTGGCTCAG (16S forward; SEQ ID NO: 1) and TACCGCGGCTGCTGGCAC (16S reverse; SEQ ID NO: 2). PCR cycling conditions included an initial denaturation at 95° C. for 30 seconds, 30 cycles of amplification (95° C. for 10 seconds, 55° C. for 20 seconds and 72° C. for 30 seconds) and a final end polishing at 72° C. for 7 minutes. PCR reactions were run using 0.1 μl of isolated genomic DNA, 1 μM of each primer and 100 μM of each dNTP in a final volume of 50 μl containing 5 μl 10X NEB Taq buffer and 0.5 μl NEB Taq polymerase.

PCR reactions were run on agarose gels to separate PCR products from unincorporated nucleotides and primers. PCR products were isolated using Qiagen Gel Isolation Kit following the standard protocol for fragments greater than 100 base pairs and greater than 4000 base pairs. PCR products were sequenced in both directions using 16S forward and 16S reverse primers, and a commercial sequencing provider (GeneWiz).

From two bacterial isolates, the following identical sequence was obtained from amplification of genomic DNA (SEQ ID NO: 3):

CTGCTGGCACGGAGTTAGCCGGTGCTTCTTCTGCGGGTAACGTCAATC
GACGCGGTTATTAACCGCATCGCCTTCCTCCCCGCTGAAAGTACTTTA
CAACCCGAAGGCCTTCTTCATACACGCGGCATGGCTGCATCAGGCTTG
CGCCCATTGTGCAATATTCCCCACTGCTGCCTCCCGTAGGAGTCTGGA
CCGTGTCTCAGTTCCAGTGTGGCTGGTCATCCTCTCAGACCAGCTAGG
GATCGTCGCCTAGGTGGGCCATTACCCCGCCTACTAGCTAATCCCATC
TGGGTTCATCCGATAGTGAGAGGCCCGAAGGTCCCCCTCTTTGGTCTT
GCGACGTTATGCGGTATTAGCCACCGTTTCCAGTGGTTATCCCCCTCT
ATCGGGCAGATCCCCAGACATTACTCACCCGTCCGCCACTCGTCACCC
AAGGAGCAAGCTCCTCTGTGCTACCGTCCGACTTGCATGTGTTAGGCC
TGCCGCCAGCGTTCAATCTGAGCCAGGATCAAACTC

Alignment of the above sequence identified the Pyrularia-associated bacteria as Pantoea ananatis, though it is noted that the exact same 16S sequence is present in Pantoea agglomerans.

### Deposit of Biological Material

The following biological material has been deposited under the terms of the Budapest Treaty with the American Type Culture Collection, 10801 University Boulevard, Manassas, Va., 20110, and given the following accession number:

| Deposit | Accession Number | Date of Deposit |
|---|---|---|
| Pantoea agglomerans | PTA-10285 | Aug. 18, 2009 |

The bacterial strain has been deposited under conditions that assure that access to the culture will be available during the pendency of this patent application to one determined by the Commissioner of Patents and Trademarks to be entitled thereto under 37C.F.R. §1.14 and 35 U.S.C. §122. The deposits represent a substantially pure culture of the deposited strain; it is noted that the strain is designated as Pantoea agglomerans for the deposit, and referred to as either Pantoea agglomerans or Pantoea ananatis herein. The deposit is available as required by foreign patent laws in countries wherein counterparts of the subject application, or its progeny, are filed. All restrictions on the availability to the public of the material so deposited will be irrevocably removed upon the granting of a patent. However, it should be understood that the availability of a deposit does not constitute a license to practice the subject invention in derogation of patent rights granted by governmental action.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An immunostimulatory composition comprising:
(i) lipopolysaccharide (LPS) isolated from Pyrularia fruit extracts; or
(ii) LPS isolated from a Pantoea species associated with Pyrularia fruit,
wherein the immunostimulatory composition is capable of activating granulocytes and/or macrophages.

2. The composition of claim 1, wherein the LPS isolated from a Pantoea species associated with Pyrularia fruit is isolated from Pantoea ananatis.

3. The composition of claim 1, wherein the LPS is substantially stable at 100° C.

4. The composition of claim 1, wherein the LPS is stable at a pH of 3-10 at 25° C.

5. The composition of claim 1, further comprising a pharmaceutically acceptable carrier.

6. A method of stimulating immunity in a subject in need thereof, comprising administering to the subject the composition of claim 1.

7. The method of claim 6, further comprising administering to the subject a therapeutically effective amount of a second immunostimulatory composition.

8. The method of claim 7, wherein the second immunostimulatory composition comprises a cytokine.

9. The method of claim 7, wherein stimulating immunity comprises inhibiting tumor development or growth in the subject, and the method comprises administering the agent to a subject having, or at risk of developing, a tumor.

10. The method of claim 9, wherein inhibiting tumor development comprises inhibiting tumor metastasis.

11. The method of claim 9, wherein stimulating immunity comprises:
(i) activating granulocytes and/or macrophages; or
(ii) inducing mitosis in an immune cell in the subject; or
(iii) both (i) and (ii).

12. A method of preparing an immunostimulatory and/or cytotoxic Pyrularia extract, comprising:
extracting Pyrularia tissue in a fluid to form a primary extract;
precipitating the primary extract with 15% ammonium sulfate to form a secondary extract; and
dialyzing the secondary extract using 12-14 kD MW cutoff dialysis tubing to produce the immunostimulatory and/or cytotoxic Pyrularia extract.

13. The method of claim 12, further comprising producing a more highly purified Pyrularia extract by
passing the immunostimulatory and/or cytotoxic Pyrularia extract over a size exclusion column; and
collecting high molecular weight fractions to form the more highly purified Pyrularia extract.

14. A method of preparing an immunostimulatory Pyrularia extract comprising:
passing the more highly purified Pyrularia extract of claim 13 over an anion exchange column; and
collecting low salt eluate from the column, to form the immunostimulatory Pyrularia extract.

15. The method of claim 14, further comprising heating the immunostimulatory Pyrularia extract to at least 60° C. for at least 10 minutes.

16. A method of preparing a cytotoxic Pyrularia extract comprising:
passing the more highly purified Pyrularia extract of claim 13 over an anion exchange column; and
collecting high salt eluate from the column, to form the cytotoxic Pyrularia extract.

17. The method of claim 12, wherein the immunostimulatory and/or cytotoxic Pyrularia extract comprises LPS from a bacterium associated with the Pyrularia tissue.

18. The method of claim 12, wherein the immunostimulatory and/or cytotoxic Pyrularia extract comprises LPS from a Pantoea bacterium associated with the Pyrularia tissue.

19. A Pyrularia extract with immunostimulatory and/or cytotoxic activity, prepared by the method of claim 12.

20. The Pyrularia extract of claim 19, wherein the extract has granulocyte-stimulatory activity.


SEQUENCE LISTING (3 sequences)

SEQ ID NO: 1 - DNA, 23 bp, Artificial Sequence
  Synthetic polynucleotide
tggagagtttgatcctggctcag

SEQ ID NO: 2 - DNA, 18 bp, Artificial Sequence
  Synthetic polynucleotide
taccgcggctgctggcac

SEQ ID NO: 3 - DNA, 516 bp, Pantoea ananatis
ctgctggcacggagttagccggtgcttcttctgcgggtaacgtcaatcgacgcggttattaaccgcatcgccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgggccattaccccgcctactagctaatcccatctgggttcatccgatagtgagaggcccgaaggtccccctctttggtcttgcgacgttatgcggtattagccaccgtttccagtggttatccccctctatcgggcagatccccagacattactcacccgtccgccactcgtcacccaaggagcaagctcctctgtgctaccgtccgacttgcatgtgttaggcctgccgccagcgttcaatctgagccaggatcaaactc